(19) United States
(12) Patent Application Publication
(10) Pub. No.: US 2010/0030132 A1
(43) Pub. Date: Feb. 4, 2010

(54) APPARATUS AND METHOD FOR WOUND, CAVITY, AND BONE TREATMENT

(76) Inventors: Jeffrey Niezgoda, Greendale, WI (US); David Correale, West Chester, PA (US); Claire Sampson, Doylestown, PA (US); David Zansitis, Downingtown, PA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/303,323

(22) PCT Filed: Sep. 28, 2007
(86) PCT No.: PCT/US07/79991

§ 371 Date: Jun. 17, 2009

Related U.S. Application Data
(60) Provisional application No. 60/847,663, filed on Sep. 28, 2006.
(60) Provisional application No. 60/956,578, filed on Aug. 17, 2007.
(60) Provisional application No. 60/970,639, filed on Sep. 7, 2007.

(51) Int. Cl. A61M 35/00; A61M 1/00; A61N 7/00; A61F 7/00

(52) U.S. Cl. 604/22; 604/289; 604/313; 604/291; 604/290

(57) ABSTRACT

The present invention provides a treatment apparatus. The apparatus contains a reservoir or generator for a treatment solution, a mechanism for delivering the treatment solution to a wound site, and a mechanism for applying the solution to a wound, tissue, bone or surgical cavity for treatment. The apparatus may apply the solution (e.g., a solution containing hypohalous acid) with, for example, an occlusive wound dressing, pulsative lavage device, hydrotherapy, hydrosurgical device, and/or ultrasound. A waste container may be operably connected to the apparatus for collecting waste from the wound by run-off, or by applying negative pressure (e.g. a vacuum). Because the apparatus of the invention can optionally be portable or mobile, the invention is suitable for use in hospitals and nursing homes, as well as for home wound care. The invention also provides a method for treating a wound (or other area needing treatment), and/or for reducing wound bioburden, by supplying a hypochlorous acid solution to the site, such as a wound colonized or infected with drug resistant bacteria, before, during, or after negative pressure wound therapy.

This application claims the benefit of U.S. Provisional Application No. 60/847,663 filed Sep. 28, 2006, U.S. Provisional Application No. 60/956,578 filed Aug. 17, 2007, and U.S. Provisional Application No. 60/970,639 filed Sep. 7, 2007, all of which are incorporated herein by reference in their entireties.

### BACKGROUND

Treating open wounds (i.e. surgical wounds, traumatic wounds, burns, venous ulcers, diabetic ulcers, arterial ulcers and decubitis ulcers) that are too large and/or infected to spontaneously close has long been a troublesome area of medical practice. Healthcare costs for wound care in the US alone are estimated at $20 billion annually. For example, chronic venous insufficiency affects approximately 2.5 million people in the United States per year with more than 500,000 people developing ulcerations. Of these cases, 50% will take greater than one year to heal thereby impacting the patients' social and economic activities. An estimated $1 billion is spent annually treating such wounds with an additional cost of $2 billion attributed to lost wages and work days. Pressure, venous stasis and diabetic ulcers are prevalent in the elderly populations and the incidence of diabetes is forecast to increase 165%, affecting up to 29 million people in the United States alone by 2050. Additionally, the growing acceptance of cosmetic surgery also presents an increased demand for improved wound treatments and accelerated wound healing.

The wound healing process is a dynamic pathway optimally leading to restoration of tissue integrity and function. Healing pathways are set into motion at the moment of wounding, and require the successive, coordinated function of a variety of cells and the close regulation of degradative and regenerative steps, including coagulation, inflammation, ground substance and matrix synthesis, angiogenesis, fibroplasia, epithelialization, wound contraction, and remodeling. These complex, overlapping processes are best organized into 3 distinct phases of healing: the inflammatory phase, the proliferative phase, and the maturation phase. Underlying this process is the proliferation, differentiation and migration of cells, fibrin clot formation and resorption, and tissue remodeling including fibrosis, endothelialization and epithelialization. Ultimately, wound healing involves the formation of highly vascularized tissue that contains numerous capillaries, active fibroblasts, and abundant collagen fibrils.

The wound healing process applies to both acute and chronic wounds. However, in chronic wounds, the sequential process of wound healing has been disrupted leading to the interruption of the normal, controlled inflammatory phase or cellular proliferative phase. Many factors can contribute to poor wound healing. The most common include local causes such as wound infection; tissue hypoxia; repeated trauma; the presence of debris and necrotic tissue; and systemic causes such as diabetes mellitus, malnutrition, immunodeficiency, and the use of certain medications. Wound infection is a particularly common reason for poor wound healing. While all wounds are contaminated with bacteria, whether a wound becomes infected is ultimately determined by the host's immune competence, the type of wound-pathogen(s) present, the formation of a microbial biofilm, and/or the numbers of bacteria present.

Infection and poor vascularization hinder the formation of granulation tissue. During the initial stages of wound healing, granulation tissue must form within the wound. Granulation tissue is a matrix of collagen, fibronectin, and hyaluronic acid carrying macrophages, fibroblasts, and neovasculature, which forms the basis for subsequent epithelialization of the wound. Particularly, a zone of stasis can form near the surface of a large open wound, where localized edema restricts the flow of blood to the epithelial and subcutaneous tissue. Without sufficient blood flow, the wound is unable heal or to fight bacterial infection. Further, biofilms and colonization by microorganisms, which may be drug-resistant, at the site of the wound can lead to frank soft tissue infection further compromising the ability of the wound to heal.

Aside from infection, a variety of other factors can influence healing of wounds. These include excessive exudate, necrotic tissue, poor tissue handling, and impaired tissue perfusion, as well as clinical conditions such as advanced age, diabetes, and steroid administration.

Exudate is a clear, straw colored liquid produced by the body in response to tissue damage. Although exudate is primarily water, it also contains cellular materials, antibodies, nutrients and oxygen. In the immediate response to an injury, exudate is produced by the body to flush away any foreign materials from the site. The exudate then becomes the carrier for polymorphs and monocytes so that they may ingest bacteria and other debris. Exudate also enables the movement of phagocytic cells within the wound and the migration of epithelial cells across the wound surface. While exudate is an important component of wound healing, excess exudate in response to chronic inflammation can aggravate a wound since the enzymes in the fluid also attack healthy tissues. Chronic wounds frequently have excessive exudate, usually associated with a chronic infection and/or biofilm that has upregulated the inflammatory cells of the body. This may be a local response or may include a systemic increase in inflammatory markers and circulating cytokines.

Chronic wounds also lead to the formation of necrotic tissue, which in turn supports the growth of microbes. Thus, initial debridement of necrotic tissue is important for wound bed preparation, so that wound treatment can progress.

The healing process and standard of care is not uniform across all types of wounds. For example, wounds resulting from ischemia, or lack of blood flow, can be difficult to heal since the decreased blood flow to the wound may prevent the normal immune reactions needed to fight infection. Patients that are bedridden or otherwise non-ambulatory are susceptible to ischemic wounds such as decubitus ulcers or pressure sores. Decubitus ulcers form as a result of constant compression of the skin surface and underlying tissue thus restricting circulation. Since the patient is often unable to feel the wound or to move sufficiently to relieve the pressure, such wounds can become self-perpetuating. Although it is common to treat such wounds with flaps, the conditions that initially caused the wound may also work against successful flap attachment. Wheelchair-bound paraplegics, for example, must still remain seated after treatment of pelvic pressure sores.

Venous ulcers occur due to improper functioning of valves in the veins, typically the legs. The standard of care for chronic venous ulcerations focuses on controlling edema and venous hypertension through appropriate compression therapy, and on reducing surface bioburden, as venous wounds typically are highly colonized.

Another type of wound is a partial thickness burn. In partial thickness bums, cell death from thermal trauma does not extend below the deepest epidermal structures such as hair follicles, sweat glands, or sebaceous glands. The progression of partial thickness bums to deeper bums is a major problem in bum therapy. The ability to control or diminish the depth of burns greatly enhances the prognosis for bum patients and decreases morbidity resulting from burns. Partial thickness bums are formed of a zone of coagulation, which encompasses tissue killed by thermal injury, and an underlying zone of stasis. Cells within the zone of stasis are viable, but the blood flow is static due to collapse of vascular structures because of localized edema. Unless blood flow is re-established within the zone of stasis soon after injury, the tissue within the zone of stasis also dies. The death of tissue within the zone of stasis is ultimately caused by lack of oxygen and nutrients, reperfusion injury (re-establishment of blood flow after prolonged ischemia), and decreased migration of white blood cells to the zone allowing bacterial proliferation.

Another type of wound is a diabetic foot ulcer. The treatment of diabetic foot ulcers is complex. The resistance of diabetic foot ulcers to healing is multifactorial and includes inadequate limb perfusion, presence of infection, and inadequate offloading. Unless all three of these factors are addressed, the wound will persist. Recognizing peripheral arterial disease is imperative and a prompt consultation by a vascular surgeon is essential to maximize healing potential. The confirmation of foot infection in diabetic patients can be difficult. About 50% or more of patients with severe diabetic foot infections have no signs of systemic toxicity. Effective wound care promotes an orderly transition from inflammation through proliferation and remodeling, and requires control of wound bioburden, since excessive bioburden can result in inflammatory and proliferative phase stagnation that compromises normal wound healing physiology. Bacterial proliferation, biofilm production, critical colonization and the development of resistant organisms can lead to infection, wound deterioration, and devastating tissue loss. Thus, therapies to control or eliminate wound bioburden while promoting normal wound healing physiology are critical for the care of chronic, severe, and/or intractable wounds.

### SUMMARY OF INVENTION

The present invention provides for complete wound or tissue care, by providing a system or apparatus to effect an orderly and/or seamless transition from wound debridement and disinfection (including asepsis), through tissue remodeling, and ultimately wound closure. The present invention provides for quicker wound closures and safer wound care environments than have been previously available. The present invention also provides for preoperative, intraoperative and post-operative treatments, including periwound, bone, tissue, intraoperative body cavity (including organ), lumen and graft irrigation and or debridement. This includes bathing or showering a patient's body prior to surgery, and preoperative cleansing and disinfecting of surrounding tissues.

The invention provides an apparatus and method for debriding, irrigating, moisturizing, cleansing, lubricating, and/or disinfecting a wound, tissue, cavity, or bone. The invention also provides a method and apparatus for reducing microbial bioburden, pain, inflammation, and odor associated with a wounded, infected, or colonized or necrotic tissue. The invention delivers chemical and/or pharmaceutical agents, such as oxidizing species, antimicrobials, growth factors and/or enzymes to the area needing treatment, and delivers such agents in a manner and/or sequence to: debride echar, exudate, necrotic tissue, and debris; cleanse, irrigate, disinfect, and remove/reduce wound bioburden and microbial biofilms; retard microbial and biofilm regrowth; decrease pain, odor, inflammation; and promote wound healing physiology.

In one aspect, the present invention provides an apparatus for infusing a wound, tissue, or cavity with a wound treatment composition, and optionally for applying negative pressure to, for example, a wound. The apparatus of the invention contains a reservoir that holds a wound treatment solution, and/or a generator that produces a wound treatment solution. The generator is an electrochemical generator for electrolyzing a salt solution, such as an ionic halide salt solution, such as a solution containing sodium chloride. The electrolyzed solution contains oxidizing species such as an ionic halide salt (e.g. HOCl, HOBr, HOI, HOF, HOAt) to debride, disinfect, and cleanse a wound, tissue, cavity, or bone. The electrolyzed solution kills wound pathogens, including nosocomial pathogens, removes/reduces wound bioburden and microbial biofilms, retards microbial and biofilm regrowth, decreases pain and odor, and promotes the physiology of healing. The apparatus of the invention further contains one or more mechanisms, in fluid communication with the generator or reservoir, for infusing a wound with the hypohalous acid solution. For example, the apparatus may have a mechanism for infusing the wound with the hypohalous acid solution in a manner that debrides, moisturizes, and disinfects the wound to bring about debridement, promotion of granulation and tissue regeneration and/or wound closure. In certain embodiments, the debriding/disinfecting is accomplished using high velocity irrigation. The debriding/disinfecting mechanism may be appropriately selected on the basis of the wound type, location, stage, and severity, and may include, for example: soak, scrub, sharps debridement, pulsatile lavage, hydrosurgery, hydrodebridement, and ultrasound. The mechanism for infusing the wound may be coupled with a wound dressing for retaining the solution around the wound, and for applying and controlling the application of negative pressure therapy, which removes waste, exudate, and necrotic tissue, increases vascular flow, and promotes formation of granulation tissue. For example, the apparatus of the invention in one embodiment infuses a wound, tissue, cavity, or bone with hypohalous acid solution in the presence of ultrasound, which will encourage microstreaming of the hypohalous acid solution into the wound tissue and host cells to promote cell proliferation, and/or microstreaming to aid in the killing of wound pathogens and removing biofilm via cavitation. In this exemplary embodiment, the apparatus may also employ a means for controlling negative pressure therapy, using the hypohalous acid solution as an irrigant. The apparatus of the invention thereby provides a means for seamless transition from wound debridement to negative pressure therapy to wound closure. These aspects of the invention further allow for the management of fluids and aerosols to control and prevent the spread of infectious microorganisms.

Because the apparatus of the invention may be plumbed directly into a wall, or may be portable or mobile, or may be embodied in table-top or cart-top units, the invention is suitable for use in hospitals and nursing homes, as well as for home wound care.

In another aspect, the invention is embodied in the form of a wound care kit, such as packaged electrolyte units and/or a wound dressing, as is described in detail herein.

In yet another aspect, the invention provides a method for debriding and disinfecting a wound, and promoting wound healing, by vigorously infusing a wound treatment solution to a wound site, e.g., using high velocity irrigation. The wound, tissue, bone or intraoperative treatment solution is a hypohalous acid solution (e.g. HOCI, HOBr, HOI, HOF, HOAt solution, or combination thereof), which may be prepared by electrolysis of a salt solution or via the mixing of chemical agents. In one embodiment, the wound treatment solution is applied by soaking and scrubbing the wound, bone or cavity and/or by one or more of pulsed lavage, hydrosurgery, and ultrasound, such that the wound, bone or cavity is irrigated and or debrided by the removal of necrotic tissue, and simultaneously disinfected. The invention may be performed before or during negative pressure wound therapy (NPWT), and/or may be performed after NPWT to promote wound healing after the end points of NPWT have been obtained. In certain embodiments, the NPWT also employs the hypohalous acid solution as an irrigating medium.

In another aspect, the invention provides a method of varying the flow and concentration of hypohalous acid in wound care solutions in a manner that is user or software selected to optimize the parameters/conditions for wound healing. In this aspect, the invention may also provide a method for monitoring the conditions of the wound by monitoring temperature, wound exudate constituents, volume of exudate. etc. for optimum wound healing.

In addition to obtaining especially short wound, tissue, and bone healing times, the apparatus and method of the invention also provide a soothing wound care to the patient to make an otherwise painful procedure more tolerable. The present invention further avoids the problem of developing resistant microorganisms at the site of the wound, as can occur with the use of traditional antibiotics and/or the development of systemic side effects such as nausea, headaches, renal or liver toxicity. Further, the present invention prevents the spread of infectious organisms to other parts of the patient's body, as well as the surrounding environment, as can be problematic with traditional methods of debridement, for example. This makes the invention particularly desirable for hospital wound care where nosocomial infections often threaten the care and overall health of susceptible patients. The present invention also overcomes drug dosage issues in patients with ischemia where, due to poor circulation and blood supply, systemically delivered drugs are decreased in potency at the site needing therapy.

### DESCRIPTION OF DRAWINGS

FIG. 1 illustrates an exemplary electrochemical cell that may be used in accordance with the invention.

FIG. 2 shows an embodiment of the invention employing a spray device with pulsed lavage for infusing a wound with an electrolyzed solution.

FIG. 3 shows an embodiment of the invention using a wound dressing and pulsed lavage with an electrolyzed solution, together with the application of negative pressure therapy.

FIG. 4 is a schematic diagram of an exemplary apparatus of the present invention employing negative pressure wound therapy.

FIG. 5 is a schematic diagram of an electrochemical generator according to one embodiment of the invention.

FIG. 6 is a cross-section side-view of an exemplary wound dressing in accordance with the invention.

FIG. 7A is a cross-section of an exemplary tube for connecting an electrochemical generator to a vacuum canister according to an embodiment of the invention. FIG. 7B is a cross-section of an exemplary tube connecting a vacuum canister to a wound dressing according to an embodiment of the invention.

FIG. 8 is a schematic diagram of an apparatus employing negative pressure wound therapy according to one embodiment.

FIG. 9 illustrates a table-top or wall mount apparatus of the invention (FIG. 10A) for convenient placement within an examination room or wound care station (FIG. 11B).

FIG. 10 illustrates an electrolyte container supplied in 3-litre units with waste management chamber (FIG. 10A), which may used in conjunction with a wound care apparatus of the invention (FIGS. 10B and 10C).

FIG. 11 illustrates alternative embodiments of the invention that accommodate IV bags (11A) and bottles of all sizes for containing the electrolyzed solution (11B).

FIGS. 12A and B illustrate an embodiment having a graphical user interface for controlling various parameters in accordance with the invention.

FIG. 13 illustrates an embodiment of the invention where the electrochemical solution (e.g., VASHE™ wound cleanser) is labeled to indicate an expiration date and to reference patient files to aid hospital reimbursement.

FIGS. 14A-E illustrate table-top or cart units of the present invention.

FIGS. 15A and B illustrate sterile bandages soaked in hypohalous solution for use as a wound dressing in accordance with the invention.

FIGS. 16A and B illustrate home-care, mobile, and field units in accordance with the present invention.

FIGS. 17A-D illustrate a system for continuous solution run-off, which may be used in conjunction with negative pressure (B) and/or ultrasound (C).

FIGS. 18A and B illustrate an applicator (A) and packaging (B) for a hypohalous acid cream or gel.

FIGS. 19A and B illustrate cart units in accordance with the invention for creating large daily batches of hypohalous acid solution.

FIG. 20 illustrates an apparatus of the invention that enables selection and attachment of complementing technologies.

FIG. 21 shows the results of vigorously applying an electrolyzed saline solution to wound patients to debride the wound, and shows the dramatic improvement in the wound size immediately upon such treatment. 142 patients were treated with an electrolyzed solution containing predominately HOCl as an active agent at the Centers for Comprehensive Wound Care and Hyperbaric Oxygen Therapy. Wound healing trajectories were analyzed prior to the initiation of HOCl therapy and then recalculated 6 weeks afterward. A marked increase in the slope of the curve (decreased wound volume) was observed, with no adverse effects.

### DETAILED DESCRIPTION OF INVENTION

### I. Wound Treatment Apparatus

In one aspect, the present invention provides a wound treatment apparatus for infusing a wound with a wound care solution, and for providing an orderly and/or seamless transition to and from negative pressure therapy. The apparatus contains a reservoir to hold a wound treatment solution and/or a generator to produce a wound treatment solution, and a mode or mechanism for applying the solution to a wound site. The apparatus of the invention debrides and disinfects chronic or intractable wounds, including full and partial thickness burns and leg ulcers, without irritation and pain and without the spread of infection. The apparatus also facilitates cell growth and regeneration thereby advancing the healing process. While the description will refer to a “wound,” the description should be construed as including applications for dermatoses, intact skin, asepsis and preoperative preparation of skin, and intraoperative and postoperative treatment of skin and tissues, grafts, and prosthetics, and implants.

The treatment solution in accordance with the invention is an electrolyzed solution of salt, or is produced via the mixing of chemical agents, to generate a hypohalous acid solution such as an ionic halide salt. e.g. a solution containing NaCl, KCl, NaBr etc. The electrolyzed solution may contain essentially a hypohalous acid as the active agent (e.g., HOCl, HOBr, HOI, HOF, HOAt), but in certain other embodiments may contain other oxidizing or radical producing species such as a hypohalite (e.g., hypochlorite ions), hydroxide, H₂O₂, O₃, singlet oxygen, O₂, and halogen-based radicals (including oxy-halogen radicals and hydroxy-halogen radicals). Additional radical species are disclosed in U.S. Pat. No. 6,878,287, which is incorporated herein by reference.

HOCl is an oxidant and biocide that is produced by the human body's natural immune system to fight infection. Specifically, invading pathogens are engulfed by neutrophils at the site of an infection or entry of the pathogen. HOCl is generated as the final step of the Oxidative Burst Pathway, with large quantities of HOCl being released into the phagocytic vesicles to destroy the invading microorganisms. It is considered, without wishing to be bound by any theory, that hypochlorous acid exerts its biocidal effect by attacking the surface and plasma membrane proteins, impairing transport of solutes and the salt balance of bacterial cells (Pieterson et al, Water S A, 22(1): 43-48 (1996)). Although HOCl is biocidal for microorganisms, it does not affect human or animal cells, at least partly because human and animal cells have extensive, highly effective defense mechanisms known as the Antioxidant Defense System (ADS). HOCl is extremely effective for killing microorganism, such as bacteria, viruses and fungal spores, yet is safe and environmentally friendly. Exogenous hypohalous acid is an attractive agent for wound therapy because it is non-hazardous, non-irritating and non-sensitizing to the skin, non-irritating to the eyes, not harmful if swallowed and shows no evidence of mutagenic activity. An added advantage is that there is no resistance or tolerance developed by the microorganisms, as occurs with the use of antibiotics. This makes the invention particularly desirable for hospital care where nosocomial infections often threaten the care and overall health of susceptible patients.

The generation of hypohalous acid solution requires only water, electricity and salt, or can be manufactured from the acidification of a hypohalite solution. For example, HOCl may be generated by passing saline solution over coated titanium electrodes separated by a semi-permeable ceramic membrane at a current of about 6 to 9 Amps. The electrolytic cell generally has separate outputs for the catholyte and anolyte, and when using an NaCl solution as the electrolyte, a solution containing HOCl is produced as the anolyte.

In certain embodiments of the apparatus, the electrolyzed solution is pre-supplied. As such, in these embodiments, the source of the electrolyte or electrolyzed solution may be a container (e.g., a bag or bottle) that stores the solution until it is demanded, or the electrolyte may be derived from an ionic salt added to mains water or from tap water containing halide ions. The solution can be prepared in advance by any suitable method or apparatus, including an electrolytic cell system described herein. Alternatively, the electrolyzed solution may be prepared using an apparatus having a self-contained electrochemical generator. The apparatus may deliver the solution to the wound or tissue continuously, and may deliver the solution at varying velocities to promote debridement, vascular stimulation, or tissue regeneration and granulation. Alternatively, or in addition, the apparatus may deliver the solution to the wound at intervals. The apparatus of the invention can deliver the solution to the wound by several debriding mechanisms, which may be interchangeable by the removal and additional of various attachable and detachable parts. These mechanisms include: soak, scrub, pulsatile lavage, hydrosurgery, ultrasound devices, and negative pressure with hypohalous acid infusion. In certain embodiments, the debriding mechanism is contained within a wound dressing to contain the solution around the wound, and to enable and control application of negative pressure therapy to the wound to promote the initial stages of wound healing, and to seamlessly advance therapy to hypohalous infusion (e.g., passive or active infusion) without negative pressure, once the endpoints of negative pressure therapy have been obtained.

For example, the apparatus of the invention may be set up for debridement of a wound with hypochlorous acid using an attachable brush, pad, sponge, bandage or the like in fluid communication with the reservoir or generator. If desired, the attachment may be an attachable ultrasonic, hydrosurgical, hydrotherapy, or pulsatile lavage device. Debridement with hypohalous acid allows for the removal of necrotic tissue and simultaneous disinfection without the risk of spreading infection to other areas of the body or surroundings, which is traditionally problematic with ultrasonic and pulsatile lavage debridement with plain saline or other traditional irrigant, as these mechanisms can cause significant splattering of bodily fluids and tissue, and aerosolization microorganisms. Alternatively, in certain embodiments, the ultrasonic or pulsatile lavage device is contained within a wound dressing to couple the infusion of solution with negative pressure therapy. In this embodiment, the apparatus of the invention offers seamless transition to negative pressure therapy with hypohalous acid infusion during or after the initial debridement. For example, the wound dressing may be functionally connected to a vacuum source and in fluid communication with the electrochemical generator. Infusion of the electrolyzed solution and the vacuum source may be under the control of a central controller to effectively coordinate these functions without disruption of the wound bed. Negative pressure therapy with hypochlorous acid infusion removes excess exudate, controls wound bioburden, and promotes vascular stimulation to effect the initial stages of wound healing including the formation of granulation tissue. Once the end-points of negative pressure therapy have been obtained, the apparatus of the invention allows for seamless transition to passive infusion or soak with hypohalous acid solution to promote continued tissue growth and regeneration until wound closure.

Various features and embodiments of the apparatus of the invention are described in more detail below.

Electrochemical Generator

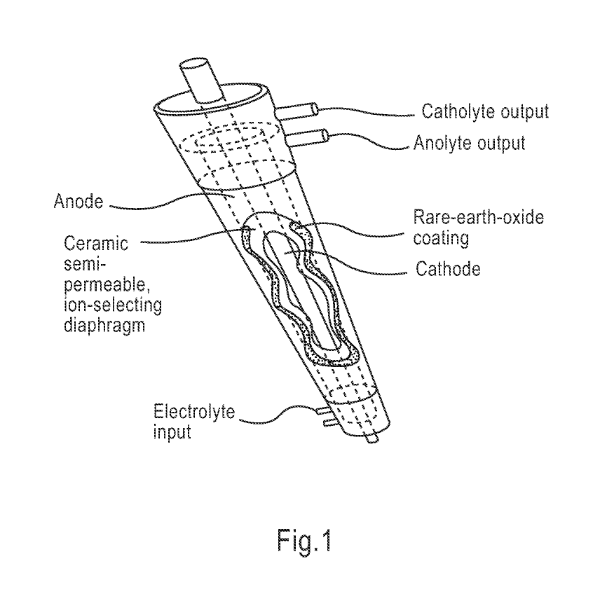

The present invention may employ an electrolysis cell such as that disclosed in US 2004/0060815, which is hereby incorporated by reference in its entirety. An exemplary cell is also illustrated in FIG. 1.

The hypohalous acid solution may be pre-supplied, that is, prepared using a stand alone electrochemical generator and added to the reservoir of the apparatus of the invention. Alternatively, the apparatus of the invention may comprise a self-contained generator for producing the electrolyzed solution, which may be delivered directly or indirectly to the wound or tissue. For example, the apparatus may optionally contain a reservoir or container for storing the electrolyzed solution until demanded, with the solution being stable for several days. The solution may be delivered from the reservoir to the wound site on demand.

Various types of electrolytic cell systems are known in the art and are suitable for use in the electrochemical generator. For example, U.S. Pat. No. 6,632,347, which is incorporated by reference herein, describes an electrolytic cell system that applies a substantially constant current across the cell between a cathode and an anode and passes a substantially constant throughput of chloride ions through the cell. The electrolytic cell system may employ any of various types of cell or electrode arrangements, including cylindrical cells, parallel discs, and parallel plate systems.

The electrochemical generator may allow for adjustment of the available free chlorine concentration (AFC) or the pH of the solution by the user. For example, the device may allow for adjustment of the solution pH by changing the chemical properties of the solution, the hydraulic regime within the electrolytic cell system, the applied electric current, or the recirculation of the catholyte. For example, the generator producing the hypohalous acid solution may control the pH of the solution using an alkaline feed from the electrolysis cell, or using conventional buffers, such as phosphate buffers stored in a separate compartment. Further, the device may allow for adjustment of the AFC content by providing a means for dilution of the resulting electrolyzed solution with an appropriate diluent, such as water or saline (e.g. 0.9% sodium chloride). Both the pH and the AFC content may be controlled or automated by the apparatus based upon the user's desired values.

In certain instances, the dispensing of the solution may be continuous at a predetermined rate or a rate determined by a controller. In other instances, the dispensing of the solution may be intermittent, which may be predetermined or determined by the controller. The solution may be dispensed using various mechanisms for propelling the solution. In certain instances, the solution may be dispensed using gravity. The gravity-induced flow of the solution can be controlled by any flow control mechanism known in the art, such as a flow control valve. In certain instances, the solution is dispensed by a pump, which can be operated to control the flow or flow rate of the solution.

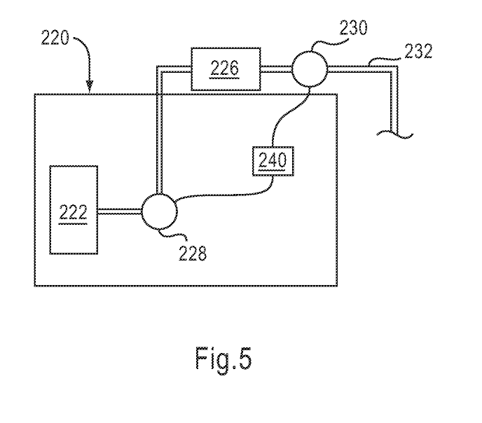

FIG. 5 shows an electrochemical generator 220 according to one embodiment. The electrochemical generator 220 comprises an electrolytic cell system 222. Electrolyzed saline solution is produced by electrolytic cell system 222 and is pumped by reservoir pump 228 into a storage container 226. When demanded, the solution is propelled through tube 232 by gravity and fluid flow is controlled by control valve 230. Both reservoir pump 228 and control valve 230 are controlled by a controller 240. Controller 240 also controls electrolytic cell system 222.

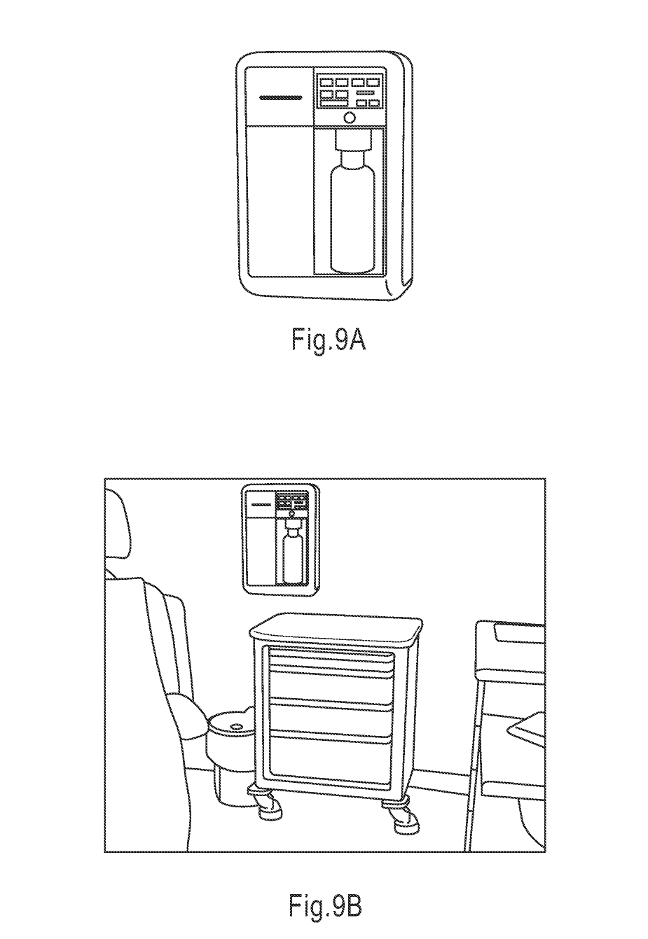

The component for generating the hypohalous solution may be of varying sizes, including sizes suitable for table-top or wall-mount devices. Such devices may be conveniently placed in an examination room or wound care station without causing obstruction of the work environment (FIG. 9). For example, the apparatus of the invention may have the following dimensions: a height in the range of 10 to 30 inches, such as 12-20 inches; a width in the range of 8 to 24 inches, such as 10-16 inches, and a depth of from 4 to 10 inches, such as from 5 to 8 inches. The generator may be powered by a battery, solar power, or through connection to an electrical outlet.

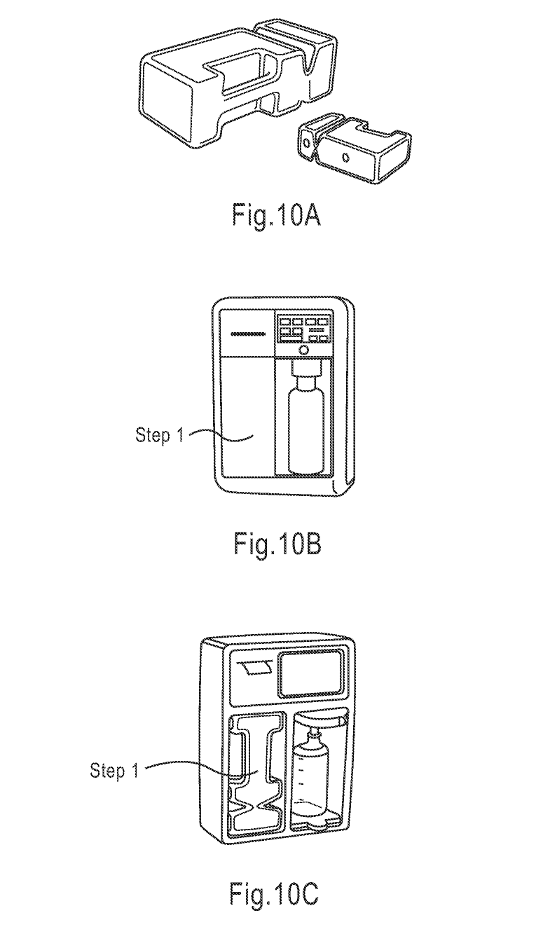

Further, dry electrolyte or dry halide salt of a single or mixture of different halide salts or liquid electrolyte (e.g., a single or mixture of different halide salts in solution) may be supplied in pre-packaged containers that allow for waste and inventory management, and which are designed for use with the electrochemical generator. For example, the electrolyte may be supplied in a dual-chamber system (FIG. 10A). The first chamber, which is placed in fluid communication with the electrochemical generator and optionally a water source, stores the dry or liquid electrolyte until needed/demanded. After electrolysis, the electrolyzed solution is applied to the wound, for example, via a wound dressing in fluid communication with the generator. The second chamber of the container is placed in fluid communication with the wound dressing and is functionally coupled to a vacuum source to apply negative pressure therapy to the wound while conveniently collecting waste for disposal in the second chamber.

The generator may be connected to main water supplies, and may further include water filters and softeners and the like, to control and maintain the purity of the water input into the system.

Electrolyzed Solution

The hypohalous acid solution generated by electrolysis of salt, such as saline, may contain a mixture of oxidizing species such as predominantly hypochlorous acid (HOCl) and sodium hypochlorite. Hypochlorous acid and hypochlorite are in equilibrium and the position of the equilibrium is determined solely by the pH (that is, pH effects the concentration of each component), which as described above may be controlled by the electrochemical generator. The electrolyzed saline solution supplied by the invention may have a pH of from 4 to 7, but in certain embodiments has a pH of from 4.5 to 6.5, or from 4.8 to 5.8, or a pH of about 5.4.

In certain embodiments, the electrolyzed solution contains essentially a hypohalous acid as the active agent (e.g., HOCl, HOBr, HOI, HOF, HOAt, or a mixture thereof), but in certain other embodiments may contain, or may also contain, other oxidizing or radical producing species such as a hypohalite (e.g., hypochlorite), hydroxide, H₂O₂ and O₃, and as described elsewhere herein.

The invention delivers a hypohalous acid solution, such as an HOCl solution, prepared by electrolysis of salt or saline, or produced via the mixing of chemical agents, and containing an available free chlorine (AFC) content or concentration of from about 5 to about 1000 parts per million. In some embodiments, the solution of the invention has an AFC content of from about 50 to about 500 parts per million, from about 140 to about 290 parts per million, or from about 150 to about 180 parts per million. The desired AFC content may be controlled by the apparatus of the invention. The purity of the hypohalous acid with respect to hypohalite is determined at least partially by the pH. For example, an electrolyzed sodium chloride solution with a pH of 5.1-6.0 has a purity of about >95% hypochlorous acid. Thus, the solution of the invention may have a purity of hypohalous acid of at least 30%, but in some embodiments, may have a purity of at least 50%, 60% 70%, 80%, 90%, 95%, or more.

The hypohalous acid solution of the invention may also contain from about 0.2 to 2.0% w/v salt, such as a halide salt, e.g. NaCl, KCl, or a mixture of salts or halide salts. In some embodiments, the invention contains 0.4 to 1.5% w/v salt, or may be a normal saline solution (0.9% w/v NaCl). The salt, or halide salt may be a salt of an alkali metal or alkali earth metal, such as sodium, potassium, or magnesium. The solution may be hypertonic, hypotonic, or isotonic with respect to physiological fluids (blood, plasma, tears, etc.). While the hypohalous solution may be delivered to the wound at room temperature, the solution may alternatively be heated, for example, to body temperature or about body temperature. In this embodiment, the solution is comfortable and soothing for the patient, and is more effective. Thus, in certain embodiments, the apparatus of the invention has a mechanism for controlling the temperature of the hypohalous acid solution being dispensed to the patient. Any heating means and means for controlling temperature, which are well known in the art, may be employed to meet this embodiment.

In certain embodiments, the electrolyzed solution is generated using a mixture of physiologically balanced salts, as disclosed in U.S. Pat. No. 6,426,066, which is hereby incorporated by reference in its entirety. Such salts may include potassium halides (e.g., KCl) and magnesium halides (e.g., MgCl₂).

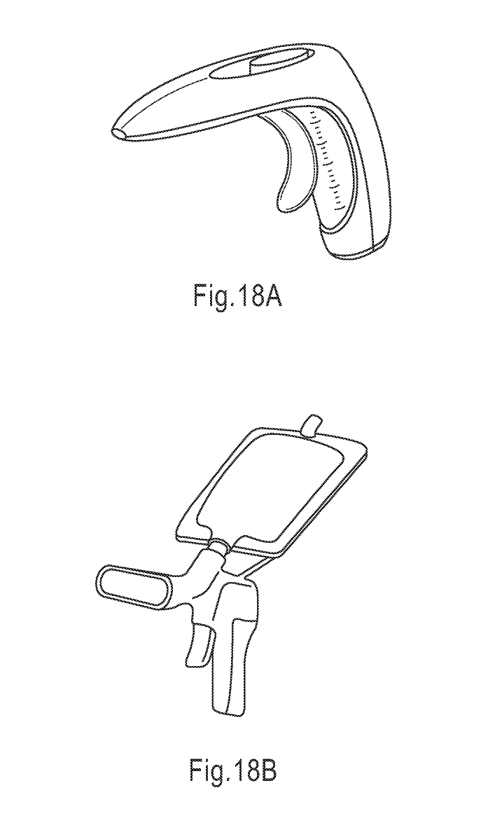

While the electrolyzed solution may be delivered in the form of a liquid, the solution may take the form of a cream, gel (e.g. silicon-based gel), and/or foam by the addition of convention additives known in the art (e.g., vitamins, aloe vera). In these embodiments, the solution is better contained around the wound site by limiting solution run-off. Further, convenient applicators for creams, foams, and the like are known, and may be used in accordance with the present invention (see FIGS. 18A and 18B).

The electrolyzed solution may have an oxidation reduction potential (redox) of between +100 mV and +1000 mV, such as greater than about +650 mV, greater than about +950 mV, such as about +1000 mV. The high redox potential allows for the quick and efficient destruction of microbes (bacteria, viruses, fungi and spores). In certain embodiments of the invention, the hypohalous acid solution delivered to the wound has a biocide rate (D Value) of approximately 1 log reduction of Bacillus subtilis spores in less than 1 minute with a 9:1 electrolyzed solution: innoculum mix. In some embodiments, the solution has a biocide rate of as low as 3.4 seconds. Generally, the hypohalous acid is effective on a broad spectrum of bacterial, fungal, and viral pathogens, including but not limited to: S. aureus (including MRSA), P. aeruginosa, E. coli, Enterococcus spp. (including, Enterococcus faecalis), Candida spp, and HIV. The invention is further effective on yeasts, Beta Haemolytic Streptococci (e.g., Streptococcus pyogenes), Serratia marcescens, Gram-positive bacteria, Gram-negative aerobic rods, Gram-negative facultative rods, including Enterobacter species, Klebsiella species, Proteus species, Anaerobes Bacteroides, Clostridium, Aspergillus, and prions. The invention is further effective on organisms resistant to antibiotics, disinfectants, and antiseptic agents.

The apparatus may, either separately or simultaneously, deliver a solution that contains a growth factor and/or debriding enzymes to promote wound healing, and may further contain an antibiotic, an odor control agent, and/or a moisturant. For example, the solution may contain proteases as well as one or more glycosaminoglycans degrading enzymes, such as, but not limited to, lysosomal hydrolases. Lysosomal hydrolases include endoglycosidases and exoglycosidases, generally acting in sequence to degrade glucosaminoglycans. Bacterial lyases may also be employed, and include: heparinase I, II and III from Flavobacteriun heparinum, which cleave heparin-like molecules; and chondroitinase ABC from Proteus vulgaris, AC from Arthrobacter aurescens or Flavobacterium heparin, and B and C from Flavobacterium heparin, which degrade chondroitin sulfate. In one embodiment, the enzyme solution is delivered prior to infusion with the hypohalous acid solution to improve debridement. In another embodiment, the enzyme or other therapeutic solution, such as an antibiotic, a growth factor, an odor control agent, a pain control agent, and/or a moisturant, is added after generation of the electrolyzed solution through an access port.

In this aspect, the present invention delivers proteolytic enzymes to the wound to help debride devitalized tissue without the necessity of surgical intervention. According to this aspect the therapeutic solution comprises an effective amount of at least one catalytically active protease selected from the group consisting of: papain, bromelain, plasminogen activator, plasmin, mast cell protease, lysosomal hydrolase, streptokinase, pepsin, vibriolysin, krill protease, chymotrypsin, trypsin, collagenase, elastase, dipase, proteinase K, Clostridium multifunctional protease, and Bacillus subtilis protease.

The invention further provides a method of delivering additional agents, such as growth factors to the wound, including oxygen, nitric oxide gas, and/or a molecules that modulate nitric oxide pathways (e.g., a PDE inhibitor). Nitric oxide has both direct and regulatory actions with known anti-infective and anti-inflammatory properties. Further, the invention includes the use of the apparatus and methods described herein in conjunction with hyperbaric oxygen therapy. These embodiments provide synergistic action with the apparatus and methods described herein for advancing therapy of wounds and tissue.

The invention may also deliver pain control medications to the wound, bone, organ, or cavity. Such anesthetics may be selected from the group consisting of benzocaine, bupivacaine, chloroprocaine, etiodocaine, lidocaine, mepivacaine, pramoxine, prilocaine, procaine, proparacaine, ropivacaine, tetracaine, dibucaine, and the pharmacologically active enantiomers thereof. Others anesthetics include morphine and pharmacologically active enantiomers thereof that interact with opiate receptors, and NSAIDS or pharmacologically active enantiomers thereof. In this aspect the present invention may deliver an analgesic, antipyretic and anti-inflammatory medication to the wound, bone, organ or cavity site. In this aspect, the solution or wound care includes the delivery of Salicylates, Aspirin, Amoxiprin, Benorilate, Choline magnesium salicylate, Diflunisal, Faislamine, Methyl salicylate, Magnesium Salicylate, Salicyl salicylate (salsalate), Arylalkanoic acids, Diclofenac, Aceclofenac, Ac emetacin, Bromfen ac, Etodol ac, Indometacin, Nabumetone, Sulindac, Tolmetin, 2-Arylpropionic acids (profens), Ibuprofen, Carprofen, Fenbufen, Fenoprofen, Flurbiprofen, Ketoprofen, Ketorolac, Loxoprofen, Naproxen, Oxaprozin, Tiaprofenic acid, Suprofen, N-Arylanthranilic acids (fenamic acids), Mefenamic acid, Meclofenamic acid, Pyrazolidine derivatives, Phenylbutazone, Azapropazone, Metamizole, Oxyphenbutazone, Sulfinprazone, Oxicams, Piroxicam, Lomoxicam, Meloxicam, Tenoxicam, COX-2 Inhibitors, and Licofelone.

Debridement and Disinfection

The apparatus of the present invention allows one or more devices to be attached in fluid communication with the electrochemical generator or reservoir, to deliver the solution to the wound site in a manner that effects wound debridement and disinfection. In some instances, the device is a simple brush or sterile pad so that the wound may be scrubbed with the hypohalous acid solution, while controlling dispense of the solution from the generator or reservoir. For example, the dispensing device may contain a button or trigger or similar control mechanism for dispensing electrolyzed solution (see FIGS. 18A, B). In these and other embodiments, more advanced debridement devices may be employed, such as hydrosurgical devices, pulsatile lavage, ultrasound and/or hydrotherapy. Such devices may be attached to the apparatus so as to be in fluid communication with the reservoir or generator of the apparatus, for example through any suitable fluid conduit, such as tubing, and by any means of physical attachment, which are well known in the art.

Debridement is a medical term referring to the removal of dead, damaged, or infected tissue or bone to improve the healing potential of the remaining healthy tissue or bone. Often this removal is surgical using a scalpel blade and is termed as sharps debridement. The present invention involves both sharps debridement and mechanical debridement of tissue or bone. Mechanical debridement in accordance with the present invention may be accomplished through vigorous application of the hypohalous acid, such as by scrub, or by pulsatile lavage, hydrosurgery, hydrotherapy, or ultrasound.

Ultrasound is sound waves vibrating at frequencies greater than what can be detected by human hearing, which is approximately 18 kilohertz (18,000 Hertz) and higher. Ultrasonic-Assisted Wound Treatment (sometimes abbreviated to UAWT) applies low-frequency power ultrasound in conjunction with an irrigation solution via a moving receptacle directly applied to the wound tissue, and can be applied as low frequency high intensity ultrasound and/or low frequency low intensity ultrasound. An ultrasonic generator transfers electric energy to a receptacle where a high-precision piezoelectric crystal transducer transforms this energy into mechanical vibrations: 25,000 vibrations/second (=25 kHz). The receptacle is continuously moved over the wound surface to loosen necrotic tissue and fibrin layers. The liquid is used for transmitting the ultrasound as well as for wound irrigation of the wound bed. The underlying mode of action of ultrasound is believed to be Cavitation, i.e. the formation and disintegration of cavities (bubbles) in liquids due to pressure fluctuations caused by the ultrasound waves (formation and subsequent implosion of these bubbles causes turbulences and changes in currents on the wound surface, which help to loosen necrotic tissue and fibrin layers at the wound site). Granulation tissue is not affected since these live cells can adapt to the pressure changes created by the ultrasound waves.

Ultrasound systems can work in the absence of a blade i.e. by cavitation (MIST technology™ (Celleration, Inc.)), and/or in the presence of a blade (i.e., by cavitation and mechanical effects) (SonicOne™, Autosonix™, and Lysonix™ (Misonix, Inc); Sonoca™ 180 (Soring, Inc.); and Qoustic Wound Therapy System™ (Arobella Medical)). When a blade is vibrated at ultrasonic frequencies and in the direction of its relatively blunt working surface, the result is a clean cutting mechanism at higher speed compared to an un-powered scalpel operated by hand. In addition, because the blade element is moving back and forth at such high speeds, nothing can stick to the blade and less force is required to cut tissues. Bone debridement can also be carried out by ultrasonics. Ultrasonic bone cutting should employ a tuned system capable of delivering sufficient acoustic power to cut hard tissue without exceeding the temperature of bone necrosis. To overcome the problem of tissue burning, ultrasonic cutting devices usually need to incorporate cooling systems, which deliver water or saline solution to the cut site.

In oral hygiene, debridement refers to the removal of the calculus that has accumulated over teeth or of damage dead or infected gum tissue. Debridement in this case can be done using hand tools and ultrasound instruments. The ultrasound dislodges the tartar. Ultrasonic bone-cutting surgery has been recently introduced as a feasible alternative to the conventional tools of cranio-maxillo-facial surgery, due to its technical characteristics of precision and safety. The device's cutting action occurs when the tool is employed on mineralized tissues, but stops on soft tissues. An example of this technology for maxofacial surgery is Piezosurgery® (reference M. Robiony, F. Polini, F. Costa, N. Zerman and M. Politi International Journal of Oral and Maxillofacial Surgery Volume 36, Issue 3, March 2007, Pages 267-269.). Other advantages include minimal risk of jeopardizing critical anatomic structures (e.g. palatine artery), minimal intraoperative bleeding and postoperative swelling, and minimal thermal damage to bone surfaces.

Incorporating hypohalous acid infusion with ultrasound in wound care in accordance with the present invention increases the speed and effectiveness of the hypohalous acid wound healing solution. This may be due to more efficient delivery of the hypohalous acid to healthy cells and microorganisms of the wound environment, promoting cell proliferation for wound healing and microbiocidal activity. Without wishing to be bound by any theory, the more efficient delivery of the hypohalous acid may be due to the micro-streaming and cavitation of the hypohalous acid by the ultrasonic device. In addition, ultrasonic debridment with hypohalous acid reduces cross-contamination/microbial contamination of the wound site and the surrounding area (as compared to the use of ultrasound without hypohalous acid). This feature is of particular importance for a hospital or wound care environments where the spread of infectious organisms must be prevented.

Ultrasonic devices that may be used in accordance with the invention include those that employ cavitation in the absence of a blade (e.g. MIST technology™ (Celleration, Inc.)), or in the presence of a blade (i.e., by cavitation and mechanical effects) (SonicOne™, Autosonix™, and Lysonix™ (Misonix, Inc); Sonoca™ 180 (Soring, Inc.); and Qoustic Wound Therapy System™ (Arobella Medical)). The present invention may further employ ultrasonic devices having designs similar to these commercially available devices. The invention may also employ an ultrasonic device as described in U.S. Pat. Nos. 5,112,300;5,180,363; 4,989,583; 4,931,047; 4,922,902; and 3,805,787, which are hereby incorporated by reference. Some ultrasonic probes include a mechanism for irrigating an area where the ultrasonic treatment is being performed (e.g., a body cavity or lumen) to wash tissue debris from the area. Mechanisms used for irrigation or aspiration may be structured such that they increase the overall cross-sectional profile of the probe, by including inner and outer concentric lumens within the probe to provide irrigation and aspiration channels for removal of particulate matter. In addition to making the probe more invasive, certain probes also maintain a strict orientation of the aspiration and the irrigation mechanism, such that the inner and outer lumens for irrigation and aspiration remain in a fixed position relative to one another, which is generally closely adjacent the area of treatment. Thus, the irrigation lumen does not extend beyond the suction lumen (i.e., there is no movement of the lumens relative to one another) and any aspiration is limited to picking up fluid and/or tissue remnants within the defined distance between the two lumens. U.S. Pat. No. 4,961,424, which is hereby incorporated by reference, discloses an ultrasonic treatment device that produces both a primary longitudinal motion, and a supplementary lateral motion of the probe tip to increase the tissue disrupting efficiency.

Literally, hydrosurgery is cutting with water. Pressurized sterile saline or water is forced under very high pressure (12800 psi to 15000 psi) through a tiny jet nozzle at the end of the hand piece producing a high velocity stream, and creating a vacuum that cuts tissue and at higher pressures will also cut bone. Water dissection works by the Venturi effect. A jet of saline, propelled by a power console, travels across the operating window of a hand-held piece and then into a suction collector. This system of pressurized saline or water functions for all intended purposes like a knife. The saline beam is aimed parallel to the wound so that the cutting mechanism is a highly controlled form of tangential excision.

More specifically, hydrosurgery may work as follows. The unit may be activated using a pedal. Sterile saline flows through low-pressure tubing to the power console where it is pressurized. Pressurized saline is forced under very high pressure through a tiny jet nozzle at the end of the hand piece, producing a high velocity stream, and creating a vacuum. This saline stream is directed backwards across the operating window and into the evacuation collector tube in the hand piece, which also collects any debris or contaminants created by the procedure. The saline and debris are collected in a waste container.

Incorporation of hypohalous acid with hydrosurgery technology increases the antimicrobial action of the hypohalous acid. Without wishing to be bound by any theory, this advantage may flow from increased penetration of the hypohalous acid into the wound tissue, to thereby effect microorganisms residing deep in the wounded tissue that would not otherwise be effected by application of a hypohalous acid to the wound site. Further, cavitation bubbles will also form within the hypohalous acid to aid in the killing of microorganisms. For example, Necrotizing Fasciitis (NF), a life-threatening condition, is characterized by bacterial infection of the skin, including the subcutaneous tissue and superficial fascia. As disclosed herein, infusion of HOCI solution to patients with NF advances the wound healing process. In addition, since hydrosurgery can be painful for the patient, hypohalous acid will actually reduce the amount of pain associated with the procedure, thereby making the procedure more tolerable. Further, use of hypohalous acid reduces cross contamination/microbial contamination, and aerosolization of bacteria during the hydrosurgery procedure, providing a safer wound care environment for all patients.

Hydrosurgical devices that may be used in accordance with the present invention include, but are not limited to, the Versajet™ (Smith & Nephew), as well as devices with similar or equivalent designs.

Pulsatile Lavage is a form of mechanical debridement using intermittent or pulsed jets of irrigant and simultaneous suction to debride a wound. This action helps to reduce the risk of infection while providing a foundation for wound healing. For use in wound management controlled irrigation pressures, specifically, irrigation pressure below 15 PSI (pounds per square inch) are typically applied to wounds. The benefits of pulsatile lavage include loosening of wound debris within a wound, reducing wound bacteria counts, debriding a wound bed and irrigating a wound when tunneling and/or undermining is present. However, patient cross contamination and aerosolization of bacteria is very problematic with pulsatile lavage therapy. An incidence of an outbreak of multidrug-resistant Acinetobacter baumannii associated with pulsatile lavage wound treatment at John Hopkin was apparently caused by dissemination of multidrug-resistant A. baumannii during the pulsatile lavage procedure, resulting in environmental contamination (JAMA. 2004;292:3006-3011).

The incorporation of hypohalous acid with pulsatile lavage, especially applied at ≦15 PSI, provides previously unrecognized advantages. First, pulsatile lavage with hypohalous acid reduces cross contamination/microbial contamination and reduces aerosolization of viable bacteria, that would otherwise occur during the pulsatile lavage procedure with other irrigants. Further, irrigation of the wound bed with hypohalous acid during pulsatile lavage increases the penetration of hypohalous acid into the wounded tissue and increases it's effectiveness as an antimicrobial and wound healing agent. Further, pulsatile lavage can also be painful, which is ameliorated by the use of hypohalous acid with the procedure in accordance with the present invention. In addition, high-pressure pulsatile lavage (20-70 psi) may be used to debride bone.

Several devices for pulsed lavage are known and available, and may be used in accordance with the present invention. These include, InterPulse™ (Stryker Inc.), Simpulse™ (Bard-Davol, Inc.), Pulsavac™ (Zimmer Inc.), and High speed pulse lavage system (MicroAire Surgical Instruments), as well as devices with similar or equivalent designs.

Negative Pressure Therapy

In certain embodiments, the apparatus of the present invention couples negative pressure therapy with infusion of a wound with an electrolyzed solution. For example, in these embodiments, the apparatus of the invention contains a wound dressing in fluid communication with the electrochemical generator or reservoir to infuse the wound and contain the wound care solution at the wound site, with the wound dressing also being functionally coupled to a vacuum source to apply negative pressure therapy. In these embodiments, infusion and negative pressure may be coordinated by a central controller, as described in detail herein. In certain embodiments, the negative pressure with the hypohalous acid solution will be applied during debridement of the wound, for example, by using a wound dressing having a self-contained debriding mechanism such as ultrasound or pulsed lavage (as described herein). Alternatively, negative pressure therapy may be applied after hypochlorous acid debridement, to promote vascular stimulation and the formation of granulation tissue. Further still, the present invention provides seamless transition from debridement to negative pressure therapy, as well as from negative pressure therapy to passive infusion with hypohalous acid, that is, without disrupting the integrity of the wound bed.

Necrotic tissue accumulates in the wound due to ongoing programmed cell death (apoptosis). In pressure ulcers for example, there are constant cycles of adequate blood flow or decreased edema cycle with periods of ischemia (from pressure) and increasing edema. Thus, the necrotic material that periodically accumulates within the wound must be removed to promote healing and prevent further bacterial growth.

Negative Pressure Wound Therapy (NPWT) promotes wound healing through several actions, which include: the removal of exudate and slough from the wound, creation of a moist wound environment, a reduction in edema, an increase in blood flow to the wound, an increase in growth factors, and the promotion of white cells and fibroblasts within the wound. Further, negative pressure brings tissue together, encouraging the tissues to stick together through natural tissue adherence. NPWT can enhance tissue perfusion, promote formation of granulation tissue, and decrease tissue edema. Functionally coupling infusion of a hypohalous acid solution with NPWT in accordance with the present invention provides unexpected decreases in wound bioburden and wound healing trajectories.

The infusion therapy of the present invention can be used in conjunction with all current NPWT devices, and delivered in either the inpatient or outpatient setting. Exemplary negative pressure devices include V.A.C.® Therapy or V.A.C.® Instill (Kinetic Concepts, Inc.) and VISTA or EZCARE (Smith & Nephew). These devices, or devices having similar or equivalent designs may be used in accordance with the present invention. Other devices for applying negative pressure to wounds, which may be used in accordance with the present invention, are disclosed in US 2007/0041960, which is hereby incorporated by reference in its entirety.

In these embodiments, wound exudate produced at the wound site is extracted from the wound dressing and/or wound site by the negative pressure device and transported away. This function may be carried out by a tube, a matrix of tubes, or other type of fluid conduit that is disposed in or on any of the layers of the wound dressing or the wound site itself. The extraction of wound exudate may be continuous, intermittent, and/or coordinated with the introduction of electrolyzed solution to optimize the fluid flow dynamics and/or efficacy of wound healing. The extraction and transportation of wound exudate may be facilitated by vacuum suctioning.

According to these embodiments, the apparatus of the invention comprises a vacuum source being functionally coupled to a wound dressing, thereby generating a negative pressure at the occluded skin lesion. For example, a vacuum canister can be in fluid communication with other components of the apparatus via one or more tubings, such as a wound dressing. The vacuum canister may operate independently or may operate under the control of a central controller. In some instances, for example, a controller may operate both the vacuum canister and the electrochemical generator and/or solution dispenser, to coordinate the operation of these components where desirable.

The vacuum canister can have any of numerous vacuum canister designs that are known in the art, such as those described in U.S. Pat. Nos. 6,695,823 and 6,142,982, which are hereby incorporated by reference. Furthermore, the vacuum canister can incorporate various features, such as pressure regulation mechanisms, filters, pressure relief valves, odor trapping mechanisms, and safety mechanisms. The vacuum canister or a portion of the vacuum canister can be adapted to be removed as necessary, such as when the vacuum canister is full of waste material. In some instances, the vacuum canister is a separate unit in the apparatus. In other instances, the apparatus comprises a self-contained vacuum canister. For example, the vacuum canister and the electrochemical generator may be combined into a single unit.

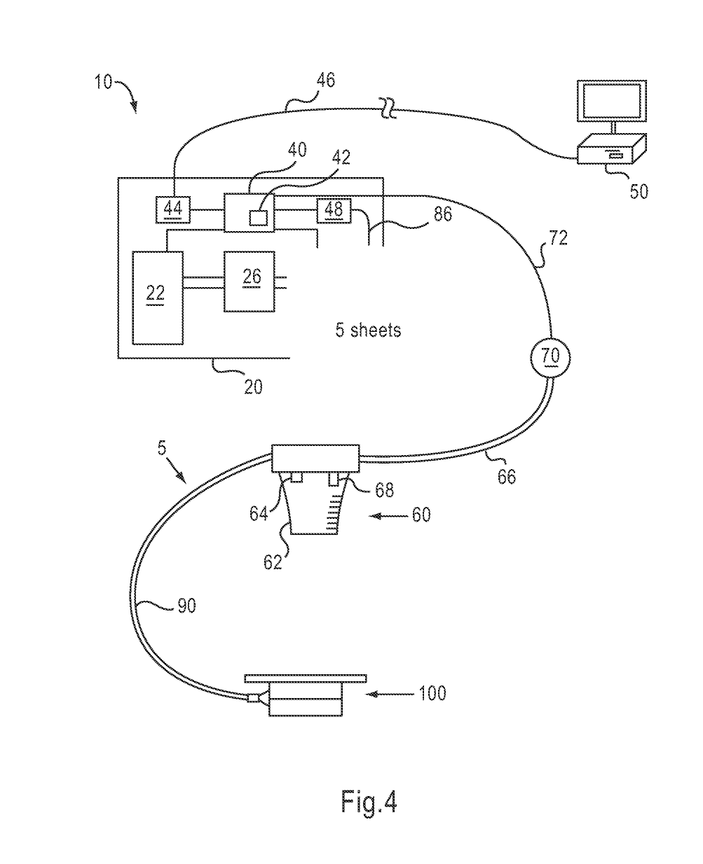

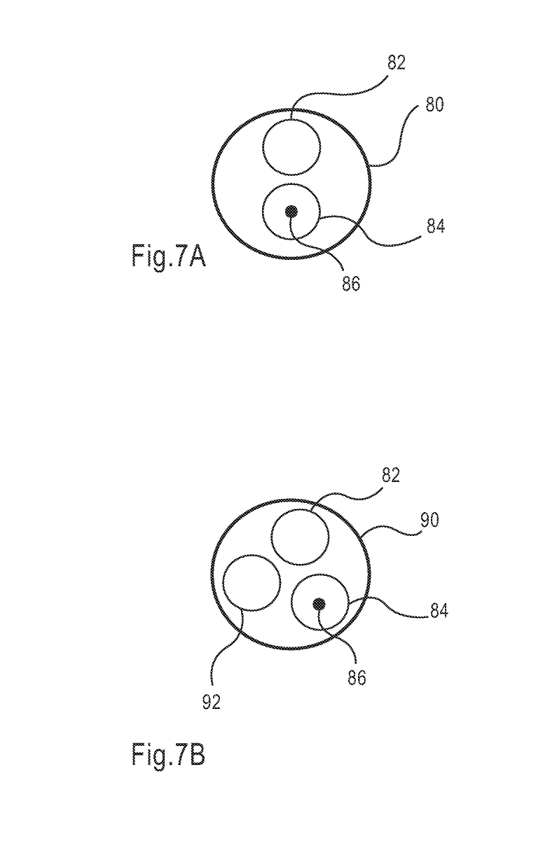

In one embodiment, as shown in FIG. 4, a vacuum canister 60 comprises a receptacle container 62, an inlet port 64, and a suction port 68. Inlet port 64 is in fluid communication with exudate drainage conduit 92 (FIG. 7). Suction port 68 is in fluid communication with a suction pump 70 and is coupled to vacuum canister 60 via a suction tube 66. In operation, suction generated by suction pump 70 is transmitted through suction tube 66, through suction port 68, through inlet port 64, and through exudate drainage conduit 92, to the wound site. The suction draws the wound exudate and transports it to vacuum canister 60 via exudate drainage conduit 92 in tube 90 (FIG. 7). Wound exudate is collected in receptacle container 62 through inlet port 64. Suction pump 70 is controlled by controller 40 via control line 72. As an additional feature, controller 40 may coordinate both the suction of wound exudate and the delivery of electrolyzed saline solution produced by electrochemical generator 20 to optimize fluid flow dynamics or efficacy in promoting wound healing.

In certain embodiments, the apparatus may further comprise a wound exudate recirculation system that allows for the recirculation of the wound exudate to the wound site. Without intending to be bound by theory, it is believed that wound exudate contains biologically active substances that promote wound healing. For example, the biologically active substances may be growth factors or cytokines that direct processes involved in wound healing. The wound exudate may be recirculated to the wound site in any of various ways. In certain instances, the wound exudate may be recirculated manually. In this case, a port for introducing and recirculating wound exudate may be provided to any of the components of the apparatus. For example, a port may be located at the source of the electrolyzed solution (e.g., one of the storage containers), in the tubings or supply conduits, or at the wound dressing. The wound exudate recirculation system may further comprise a filter for filtering out debris, bacteria, or other contaminants from the raw wound exudate. In some cases, a filter may not be necessary. For example, when using an electrolyzed solution in accordance with the invention, the bacteria are generally killed while dwelling in the biocidal solution, thus rendering bacteria filtering unnecessary.

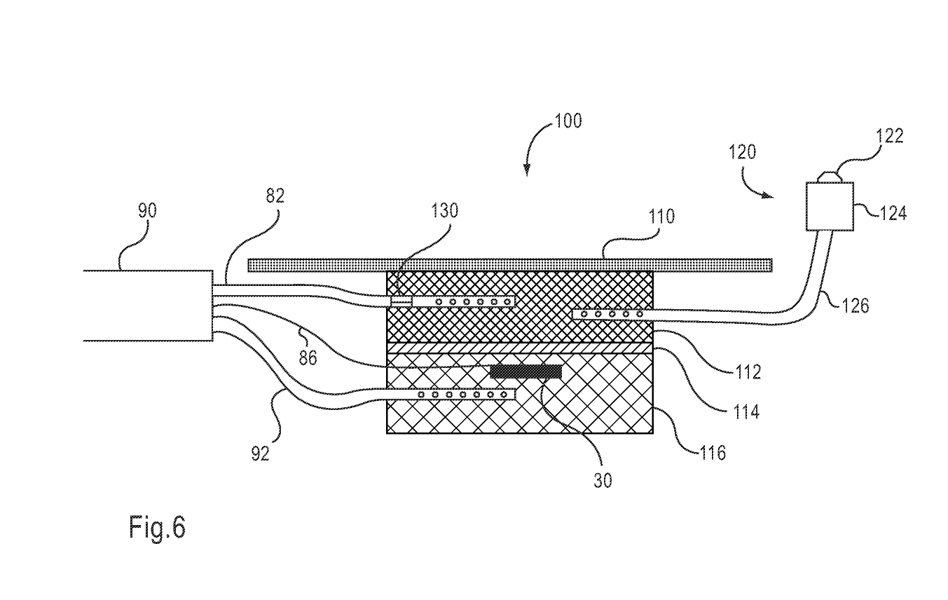

In an embodiment, as shown in FIG. 6, an exudate recirculation extension 120 is provided for wound dressing 100. Exudate recirculation extension 120 comprises an injection port 122, a filter 124, and an injection tubing 126. The distal end of injection tubing 126 is disposed in reservoir layer 112. In operation, wound exudate is drawn into a syringe and then injected through injection port 122. Debris, bacteria, and other contaminants are filtered-out by filter 124. The wound exudate then travels through injection tubing 126 and is delivered to reservoir layer 112, where it is recirculated to the wound site.

In other instances, the wound exudate recirculation system may be automated. The automated system can comprise a pump for drawing up the wound exudate and recirculating it into the electrolyzed saline solution being delivered to the wound dressing. For example, the wound exudate may be drawn from the vacuum canister. One of ordinary skill in the art can readily design such an automated system using various tubings, connections, valves, ports, pumps, and other components that are generally used in fluid transfer and hydraulics.

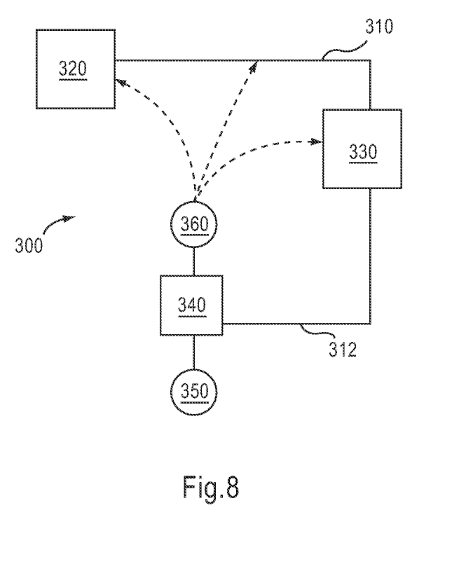

In an alternate embodiment, as shown in FIG. 8, an apparatus 300 comprises a source of electrolyzed solution 320, a wound dressing 330, supply conduit 310, drainage conduit 312, and vacuum canister 340. Electrolyzed solution is provided to wound dressing 330 via supply conduit 310. Facilitated by a suction pump 350, wound exudate is extracted from wound dressing 330 and drained into vacuum canister 340 via drainage conduit 312. A recirculation pump 360 withdraws wound exudate from vacuum canister 340 and recirculates it. As shown by the broken arrows, the wound exudate may be recirculated into the electrolyzed solution at various points in apparatus 300, including source 320, supply conduit 310, or wound dressing 330.

Wound Dressings

The apparatus of the invention may comprise a wound dressing for functionally coupling infusion of a hypohalous acid solution with debridement and/or negative pressure therapy. For example, the wound dressing may be adapted for various wound care functions, including negative pressure treatment, absorption of wound exudate, infusion of hypohalous acid solution, pulsatile lavage, ultrasound, and/or recirculation of wound exudate.

In certain embodiments, the wound dressing comprises an outer barrier layer. The outer barrier layer may serve various functions, including for example, providing a barrier against the loss of fluid or moisture from the wound dressing, securing the wound dressing to the patient, protecting the wound site from injury or contamination, or providing a seal that allows negative pressure to be maintained over the wound site.

In certain instances, the outer barrier layer may be designed in a manner similar to the drapes or backings that are used for wound dressings, bandages, or patches. The outer barrier layer can be made of any material that is semi-permeable or impermeable to fluid or moisture. Various types of non-porous polymer or plastic materials are suitable for use in the outer barrier layer. For example, the outer barrier layer may be formed of a MYLAR®/hydrocolloid combination. The outer barrier layer may be flexible, elastic, or rigid, depending upon the application and the location where the wound dressing is to be used. In certain instances, the outer barrier layer is made of an elastomeric material.

In certain instances, the outer barrier layer functions to secure the wound dressing to the patient and/or provide a seal for negative pressure treatment. Various means are available for securing the wound dressing and/or forming a seal. In some instances, the outer barrier layer may have a larger footprint than the other layers in the wound dressing. The extra surface area can have an adhesive material on one side to facilitate adhering of the outer barrier layer to the patient around a wound site. In other instances, negative pressure applied within the outer barrier layer may be sufficient for adhering the outer barrier layer to the patient. In such instances, the outer barrier layer may have a non-adhesive material, such as any suitable sealant material.

In some instances, the outer barrier layer may be sized and shaped such that it can be wrapped around a body part, such as a leg or arm, and held in place by any suitable securing means such as Velcro, wires, clips, or other fastening mechanisms. In some instances, the drape may be secured by a support wrap, such as an overlying elastic garment. In some instances, the outer barrier layer may have one or more openings to admit tubings, ports, transmission lines, or other fittings or connections with the wound dressing.

In certain embodiments, the wound dressing comprises a reservoir layer that serves as a reservoir for the electrolyzed solution. The reservoir layer is designed to absorb and temporarily retain the electrolyzed solution until it is administered to the wound site. The reservoir layer may optionally be coupled to an electrochemical generator as described herein. The reservoir layer may be formed of any suitable material that is capable of serving its function. For example, the material may be a hydrophobic material such as polyurethane ester constructed as an open-cell, foam material. In another example, the material may be a hydrophilic material such as polyvinyl acetate or hydrophilic polyurethane, which can be constructed as a small, closed-cell foam. The degree of hydrophobicity or hydrophilicity can vary depending upon the particular application of the wound dressing. The architecture of the reservoir layer can also vary depending on the application. For example, the reservoir layer may be porous (with pores that are open or closed), foam, woven, non-woven, a fine mesh, or have a fibrous scaffolding.

The electrolyzed solution may be introduced into the reservoir layer via a tube, a network of tubes, or an irrigation system which is adapted to distribute the solution. The electrolyzed solution may be provided to the reservoir layer at various flow rates or using various flow patterns. For example, the flow rate may be lower when the reservoir is saturated and higher when the reservoir level is low, and this flow rate may be controlled automatically by a central controller. In another example, the electrolyzed solution may be provided intermittently.

The wound dressing may further comprise a contact layer which is in contact with the wound site or tissue. The contact layer is designed to absorb wound exudate from the wound site and/or distribute the electrolyzed solution to the wound site. As such, the contact layer may be made of any suitable fluid or moisture-absorbent material. In certain instances, the absorbent material may be capable of wicking up wound exudate by capillary action. Examples of materials suitable for use in the contact layer include those materials that are used in sponges, dressings, bandages, diapers, and tampons. More specifically, suitable materials include natural (e.g., cellulose or cotton) or synthetic fibers (e.g., nylon, polyester, or rayon), polymeric foams (e.g., polyurethane), and absorbent hydrogels or hydrocolloids. Hydrogels are well known in the art and can be made from any of various materials such as polyvinyl alcohol, poly(N-isopropylacrylamide), chitosan, guar gum, dextran, or pectin. The architecture of the contact layer can vary depending on the application. For example, the contact layer may be porous (with pores that are open or closed), foam, woven, non-woven, a fine mesh, or have a fibrous scaffolding. Any portion of the contact layer can also be made of a biodegradable material.

In certain embodiments, the wound dressing further comprises a flow regulating layer disposed between a reservoir layer and a contact layer. The flow regulating layer is designed to regulate the flow of electrolyzed solution from the reservoir layer to the contact layer. For example, the flow regulating layer may limit the flow to a certain maximum rate. The flow regulating layer may further be designed to inhibit the flow of wound exudate in the reverse direction (i.e., from the contact layer to the reservoir layer). As such, the flow regulating layer may be any of various types of films, membranes, or layers that regulate the flow of vapor or fluid. For example, the flow regulating layer may be a moisture vapor layer that is relatively impermeable to liquid, but permeable to vapor. The flow may be regulated by the vapor or fluid pressure on one or both sides of the moisture vapor layer.

Examples of suitable materials for the flow regulating layer include various polymeric materials, such as polyurethanes (e.g., ESTANE®, manufactured by B.F. Goodrich), polyether-amide block copolymers (e.g., PEBAX®, manufactured by Elf Atochem), and polyether-ester block copolymers (e.g., HYTREL®, manufactured by DuPont). The flow regulating layer may be made of one or more types of monomers (e.g., copolymers) or as blend of polymers. Various factors will determine the permeability and flow regulatory properties of the flow regulating layer, including its composition, porosity, architecture, and thickness.

In certain instances, the wound dressing may be designed so that regional control of fluid or vapor flow is possible. For example, fluid or vapor permeability in one region of the flow regulating layer may be regulated without affecting permeability in other areas. In some instances, the wound dressing may be configured so that there is less fluid flow in the outer periphery of the wound dressing than in the central regions of the wound dressing. For example, a flow regulating layer may be used to cover the outer edges or side-walls of the wound dressing. In another example, the outer periphery of one or more layers of the wound dressing may be made from a different material than the central regions of the layer. This feature may be useful where it is desirable to reduce moisture in the periphery of the wound site, while maintaining moisture in the central regions of the wound site.

Any of the various layers in the wound dressing may further comprise an antimicrobial agent. For example, the contact layer may be impregnated with an antimicrobial silver compound within the interstices or pores of the absorbent material.

FIG. 6 shows an exemplary wound dressing 100 according to an embodiment of the present invention. Wound dressing 100 comprises an outer barrier layer 110, a reservoir layer 112, moisture vapor layer 114, and a contact layer 116. The distal end of a supply conduit 82 is disposed in reservoir layer 112 and electrolyzed saline solution is delivered to reservoir layer 112 through openings at the distal end of supply conduit 82. Supply conduit 82 also includes a one-way valve 130 that permits reservoir layer 112 to maintain positive fluid pressure. The electrolyzed saline solution is transmitted through moisture vapor layer 114 to absorbent layer 116, where it is then distributed to the wound site. The proximal end of an exudate drainage conduit 92 is disposed in contact layer 116. Wound exudate is extracted from contact layer 116 through openings at the proximal end of exudate drainage conduit 92.

Wound dressing 100 may be irrigated with electrolyzed solution in various ways. In one example, a certain amount of electrolyzed solution is delivered to reservoir layer 112. The flow of solution is then stopped and the electrolyzed solution is allowed time to transfer into contact layer 116 and be distributed to the wound site. After a certain dwell time, vacuum suction is applied through exudate drainage conduit 92 to extract the wound exudate. After a certain time interval for wound exudate extraction, electrolyzed solution is introduced again into reservoir layer 112 and the cycle is repeated.

In another example, where it is desirable to reduce moisture in the periphery of the wound site while maintaining moisture in the central regions of the wound site, the irrigation system may deliver the electrolyzed solution to the central portions of reservoir layer 112. Alternatively, where it is desirable to distribute the electrolyzed solution more uniformly, the irrigation tubing may take a zigzag course through reservoir layer 112.

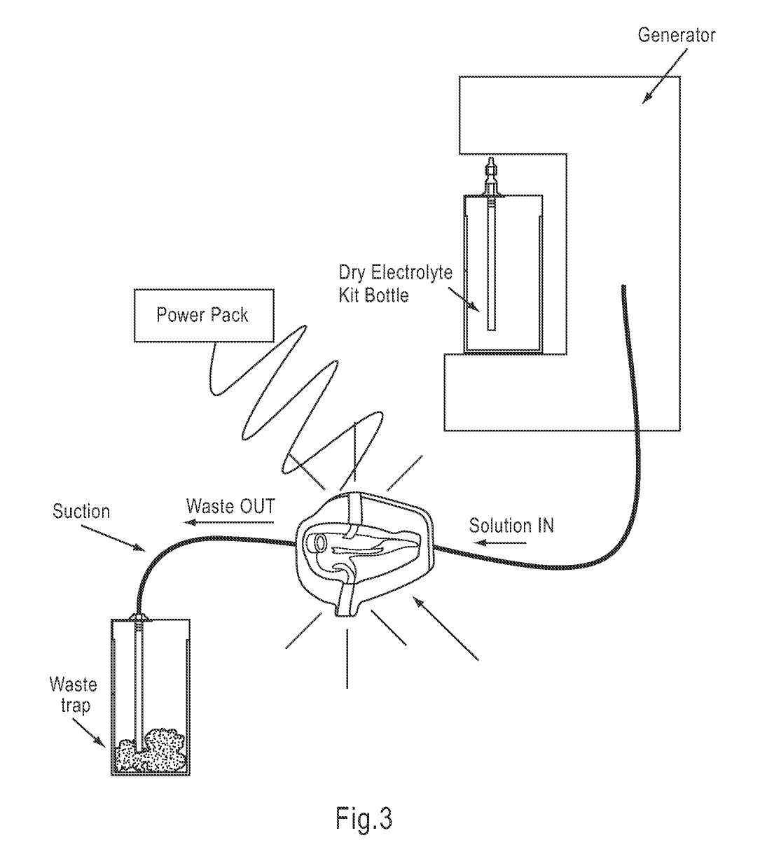

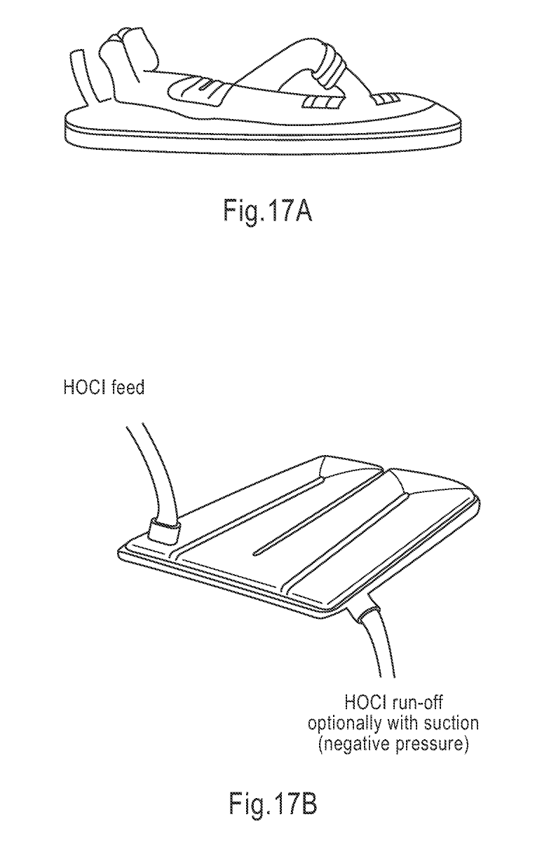

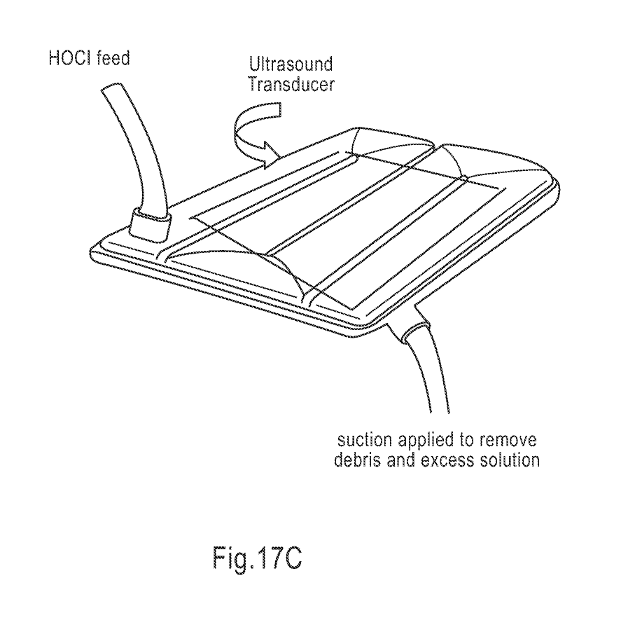

In certain embodiments, the wound dressing further contains a means for pulsed lavage (FIG. 3) or an ultrasound transducer to involve inertial acoustic cavitation (FIG. 17). This embodiment functionally couples debridement, disinfection, NPWT, and passive infusion, thereby providing complete and seamless wound care from initial debridement to wound closure. In such embodiments, the wound dressing of the invention may be designed to allow for continuous supply and run-off of the hypohalous acid solution by employing a feed conduit for infusion, and a run-off conduit, which may employ suction (negative pressure) (FIGS. 17B).

Sensors and Controls

In certain embodiments, the apparatus may further comprise one or more sensors to monitor the condition of the wound site, wound exudate, and/or the wound dressing. The sensors may be located in various components of the apparatus. In some instances, a sensor may be located in the wound dressing. For example, a sensor can be positioned in the reservoir layer, flow regulating layer, contact layer, or between any of these layers. The sensor may sense any of various conditions in the wound site, wound exudate, and/or the wound dressing. For example, the sensor may sense the pH of the fluid in the wound dressing. Various pH sensors are known in the art, including those that operate by using electrodes and those that operate using optical sensing of color change in a pH indicator. In other examples, the sensor may sense any of other factors that indicate the condition of the wound site, wound exudate, and/or wound dressing, such as temperature, nitric oxide level, fluid flow rate, moisture level, fluid pressure, or the electrical conductivity of the fluid.

In some instances, the sensor may be located in the vacuum canister. For example, the sensor may measure the volume of exudate that has been collected into the vacuum canister, or any of the above described parameters.

The sensors may be in communication with a controller. The communication link between the sensor and the controller may be through a transmission line such as a wire or fiber optic cable, or may be wireless, such as infrared or radio. In some instances, the sensors may communicate with the controller via a digital signal processor to convert analog signals into digital form.

In an embodiment, as shown in FIG. 6, a pH sensor 30 is disposed in an absorbent layer 116 of wound dressing 100. The pH sensor 30 sends signals to digital signal processor 48 via transmission line 86. The digital signals are then transmitted to controller 40.

In certain embodiments, the apparatus further comprises a controller for controlling the operation of various components of the apparatus, such as the electrochemical generator, vacuum source, wound dressing, ultrasonic or pulsatile lavage device, or the various pumps or valves in the apparatus. The controller may comprise a microprocessor, as well as other components that generally form microprocessor computer systems, such as memory, instructions (as either hardware or software), I/O bridges, buses, etc. The controller may be in communication with and control various components of the electrochemical generator. For example, the controller may control the electrolytic cell system, or any of the pump or valve components. The controller may be a separate unit or may be combined with another component of the apparatus. For example, the controller may be incorporated into the electrochemical generator to form a single unit.

The controller may also be in communication with and receive input from a sensor. In certain instances, the controller can control the operation of the electrochemical generator based on the input signals from the sensor. For example, the controller may receive input signals from a pH sensor in the wound dressing indicating that the pH has drifted beyond a predetermined range. In response, the controller can operate the electrochemical generator to adjust the amount of flow or the pH of the electrolyzed solution being delivered into the wound dressing.

In certain instances, the controller may be pre-programmed with instructions for operating the electrochemical generator in a pre-determined fashion. For example, the controller may be pre-programmed to operate the electrochemical generator to initially provide a solution having a higher concentration of hypohalous acid. Over time, the concentration of hypohalous acid is gradually reduced. This feature may be useful in optimizing the efficacy of the apparatus in promoting wound healing.

In an embodiment, as shown in FIG. 4, electrochemical generator 20 includes a controller 40, which comprises a microprocessor 42 (FIG. 5). The controller is in communication with electrolytic cell system 22, a dispensing pump, suction pump 70, modem 44, and sensor 30 (shown in FIG. 6) via digital signal processor 48. Controller 40 receives signals from sensor 30 as processed by digital signal processor 48. In response to the information conveyed by sensor 30, controller 40 operates the dispensing pump and/or electrolytic cell system 22 according to a set of program instructions. For example, with respect to the dispensing pump, controller 40 may activate/deactivate the pump or increase/decrease pumping speed. In another example, with respect to electrolytic cell system 22, controller 40 may increase/decrease electrical current in the electrodes or increase/decrease the flow of catholyte recirculation in the system. Via modem 44, controller 40 may communicate with external computer systems. Controller 40 may also coordinate the activity of dispensing pump and suction pump 70 to control the fluid dynamics in wound dressing 100.

In certain embodiments, the apparatus may further comprise a remote computer system that is in communication with the controller via a communication link. The remote computer system is located at a remote site, such as another room, another building, or even a location many miles away from the location of the controller. The communication between the controller and the remote computer system may be facilitated by various types of hardware components (e.g., modems) and/or software.

The communication link between the controller and the remote computer system may be through any suitable medium, such as telephone lines, cable lines, DSL, fiber optic lines, or other transmission lines. The communication link may also be wireless, such as radio, infrared, or microwave. The communication link may employ any of various communication protocols, including protocols over the Internet or through any of various LAN architectures and protocols, such as Ethernet and TCP/IP.

Various types of messages may be sent and received between the controller and the remote computer system. In certain instances, the controller may relay message containing data to the remote computer system, which in response, returns messages containing instructions for controlling the operation of the apparatus. For example, the controller may send messages that contain data relating to the status of the electrochemical generator and/or the wound dressing, such as the pH of the wound dressing fluid. The remote computer system may receive the messages, and in response, return a message containing instructions for operating the electrochemical generator to adjust the amount of flow or the pH of the electrolyzed solution being delivered into the wound dressing. This feature allows for the main body of the apparatus to be located in a patient's home or a nursing home, while being monitored and controlled from a remote location such as a hospital, physician's office, or other type of treatment center.

In certain embodiments, the healthcare worker or patient using the apparatus and method of the invention downloads a tree-dimensional image (e.g., point cloud of data) of the wound to/through the controller for communication transmission. The data collection may include spectro-photometry (colormetrics) for diagnostic purposes.

In an embodiment, as shown in FIG. 4, a remote computer system 50 is located at a remote site. Controller 40 is in communication with remote computer system 50 using modem 44 via telephone line 46. Controller 40 sends outgoing messages to remote computer system 50. The message may contain data relating to signals received from sensor 30 and/or the status of electrochemical generator 20. Remote computer system 50 sends return messages to controller 40, wherein the messages contain instructions to controller 40 for operating electrochemical generator 20.

### II. Wound Treatment Kits

In other aspects of the invention, wound treatment kits are provided.

In one embodiment, the wound treatment kit comprises a container for electrolyte having a plurality of chambers as described herein. For example, the first chamber contains dry electrolyte or electrolyte in solution, and one or more openings, inlets, or valve systems for placing the chamber (and the electrolyte) in fluid communication with an electrochemical generator and water source if necessary (FIG. 10A). A second chamber of the container is empty, and has one or more openings, inlets, or valve systems that may be functionally connected with a vacuum source and a wound dressing to collect waste from a wound site, as described herein. That is, when positioned in an apparatus of the invention having an electrochemical generator (FIGS. 10B and C) and vacuum source, the container creates a closed-loop system in which electrolyzed solution is prepared from the electrolyte in the first chamber via the connections to an electrochemical generator (and water source where dry electrolyte is supplied), and delivered to a wound dressing. Waste from the wound is then collected by suction via the vacuum source and stored in the second chamber for easy disposal. Where the electrolyte is in solution, the solute is typically a saline solution suitable for medical applications (e.g., normal saline). The container may be of varying sizes, such as from one to ten liters, or from two to five liters. In one embodiment, the container holds about 3 liters of electrolyte. The container may be made of any suitable inert material, such as a plastic or glass. The electrolyte may be any suitable electrolyte for wound care as described herein, such as sodium chloride or a mixture of salts so as to produce an electrolyzed solution that is physiologically balanced.

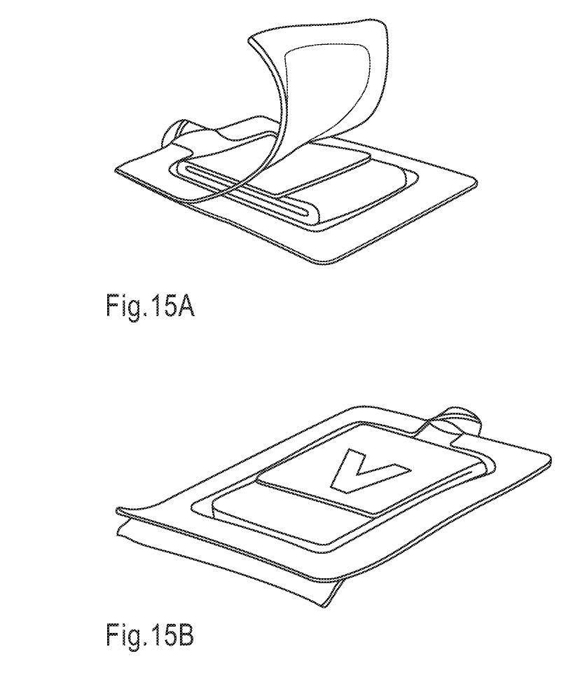

In another embodiment, the kit of the invention comprises a packaged sterile wound dressing, where the package may be operably connected to a fluid source. In this embodiment, the packaged wound dressings may be soaked with hypohalous acid solution prior to use without compromising the integrity of the packaging. For example, freshly prepared hypohalous acid solution may be injected into the packaging using, for example, a syringe or other suitable or equivalent mechanism. The package may employ any suitable locking connector port, which are well known in the art (e.g., a Luer Lock port), to allow the package to be filled with electrolyzed solution without compromising the integrity of the package (FIGS. 15 A, B). Thus, the kit may comprise packaged sterile wound dressings such as sterile gauze or other bandages, and one or more syringes that may be operably connected to the package for delivering an HOCl solution to the package. The package may be heat sealed, or sealed in any other equivalent means, which are well known in the art. The packaged wound dressing of this embodiment may be supplied in a kit with electrolyte units for creating an electrolyzed solution, such as the electrolyte units described herein.

In another embodiment, the kit of the invention comprises a wound dressing, as described herein, which provides for a continuous infusion of hypohalous acid solution together with negative pressure therapy. The wound dressing may comprise an ultrasonic transducer for debriding the wound via ultrasound, thereby involving inertial acoustic cavitation. Alternatively, the wound dressing may have a mechanism for delivering the solution by pulsed-lavage. The ultrasonic or pulsatile lavage mechanism may be of any suitable design known in the art or as described herein. In one embodiment, the wound dressing may comprise a feed inlet and a run-off outlet. The inlet may be functionally connected via any suitable connection or fluid conduit (such as tubing) to a hypohalous acid solution source. The outlet may be functionally connected to a vacuum source so as to remove waste such as exudate and necrotic material and/or excess solution from the wound. (FIG. 17B). The wound dressing further comprises at least two layers, a contact and/or reservoir layer allowing for controlled infusion of hypohalous acid solution, and an outer barrier layer as describe herein. Alternatively, the wound dressing of the invention, as illustrated in FIG. 6, may comprise a plurality of layers, including an outer barrier layer, a contact layer, and a fluid regulating layer, as described herein.

In certain embodiments, the wound dressing may also employ a sensor for monitoring certain conditions of the wound, treatment solution, or exudate, as described herein.

The wound dressing of the invention may be supplied in a kit along with electrolyte units, tubings, and a vacuum canisters as described herein, for functionally connecting the wound dressing to an apparatus as described herein.

### III. Treatment Methods

In another aspect, the invention provides a method for treating an infected or colonized wound, tissue, surgical cavity, or bone, and a method for reducing wound bioburden. The treatment solution in accordance with the invention, as already described, is generally effective for killing or inactivating a broad spectrum of bacterial, fungal, and viral pathogens, including S. aureus, P. aeruginosa, E. coli, Enterococcus spp., and Candida Spp, as described herein. The treatment solution of the invention does not produce resistant species, making the method of the invention desirable over the delivery of traditional antibiotics. Thus, in some embodiments of the invention, the patient is not being administered an antibiotic(s).

This aspect of the invention is effective for killing or inactivating bacteria, such as Staphylococcus aureus at the site of a wound or tissue, including drug-resistant staph Staphylococcus aureus is a bacteria often found on the skin of healthy people. The organism can cause infections, including minor infections such as pimples and boils, as well as more serious wound or bloodstream infections. Some staphylococcus bacteria have become resistant to antibiotics. “MRSA” refers to staph that is resistant to beta-lactam antibiotics, which include methicillin, penicillin, oxacillin and amoxicillin. The invention reduces the amount of such bacteria colonizing or infecting a wound site by from 3 logs to 7 logs, such as a 5-log reduction or a 6-log reduction in staph. In one embodiment, the invention delivers a hypohalous acid solution to the wound, such as a HOCl solution, which has a broad antimicrobial spectrum.

For example, the invention may be applied to a wound colonized or infected with drug resistant S. aureas, such as beta-lactam resistant S. aureas, or another drug-resistant microorganism against which hypohalous acid is effective. The invention may achieve a reduction of about 6 logs in under 1 minute, or in about 30 seconds. In this embodiment, the hypohalous acid solution may have an available free chlorine (AFC) content of from 5 to 1000 ppm, such an AFC content of from 100 to 200 ppm.

The method comprises supplying a wound treatment solution, such as a hypohalous solution as described herein, to a wound site by one or more of soak, scrub, pulsed lavage, hydrosurgery, and ultrasound as described herein to effectively debride and disinfect a wound or tissue. The wound site may be colonized or infected with drug-resistant bacteria, such as MRSA. In some embodiments, the solution delivered by the above techniques, or by the apparatus of the invention, may also be contained around the wound site with a wound dressing as described herein during the application of pulsed lavage or ultrasound. The solution may be delivered before, during and/or after negative pressure wound therapy (as described herein) to promote proper wound healing physiology. In these embodiments, the method may employ a wound dressing for coordinating debridement by infusion of hypohalous acid with negative pressure therapy. Thus, the method of the invention may be performed by employing the wound treatment apparatus, wound care solution, and/or wound dressing described herein.

For example, the method of the invention allows for an initial hypohalous acid soak and/or scrub to both debride and disinfect the wound or tissue, followed by the application of negative pressure to the wound or tissue (as described herein) using the hypohalous acid as an irrigant to control wound bioburden, remove excess exudate, and promote formation of granulation tissue. Optionally, the method also involves seamless transition to hypohalous acid infusion (e.g., active or passive infusion without negative pressure). Such seamless transition can be effected via a wound dressing as described herein which allows for controlled infusion of hypohalous acid solution with controlled vacuum source. In this embodiment, continued cell proliferation and regeneration continues without disruption of the wound bed, once the endpoints of negative pressure therapy have been obtained.

In another embodiment, the invention involves decontaminating the peri-apical wound area with hypohalous acid solution in advance of applying a wound dressing, thereby reducing bioburden, including the presence of MRSA. In this embodiment, the solution may also be delivered by pulsed lavage, hydrosurgery, or ultrasonics as described herein, in the absence of negative pressure therapy.

The present invention is generally applicable for the controlled disinfection, debridement, and treatment of wounds, including, but not limited to, stage I-IV pressure ulcers, stasis ulcers, and chronic open wounds such as decubitus ulcers and diabetic ulcers. In these and other embodiments, the wound may be a chronic venous ulcer (e.g. chronic venous leg ulcer), necrotizing fasciitis, or a post-surgical wound. In another embodiment, the wound is a full or partial thickness bum. In certain embodiments, the burn has been treated by tangential excision or skin graft prior to treatment in accordance with the present invention.

The present invention also provides a method for treating osteomyelitis, by employing the apparatus or methods described herein. Osteomyelitis is an infection of bone, which can either be acute (of recent onset) or chronic (longstanding), and like infected wounds, benefits from irrigation, debridement, disinfecting, and cleansing with chemical and/or pharmaceutical agents, such as oxidizing species, antimicrobials (as described herein) to remove the dead and infected bone. Bacteria are the usual infectious agents. The two likely access methods are by primary infection of the bloodstream (including secondary infection via the blood following an infection somewhere else in the body), and a wound or injury that permits bacteria to directly reach the bone. In adults, the pelvis and the spinal vertebrae are most vulnerable, while bone infections in children tend to target the long bones of the arms and legs. Without treatment, the infection and inflammation block blood vessels. The lack of oxygen and nutrients cause the bone tissue to die, which leads to chronic osteomyelitis. Other possible complications include blood poisoning and bone abscesses. Treatment options include intravenous and oral antibiotics, and surgical draining and cleaning and debridement of the affected bone tissue.

In most cases, the micro-organisms are bacteria such as Staphylococcus aureus, but fungi and viruses can also cause osteomyelitis. Extremely rarely, the viruses which cause chickenpox and smallpox have been found to cause a viral osteomyelitis. Some of the conditions and events that can lead to osteomyelitis include: bacteria introduced during bone surgery, bacteria introduced by trauma to bone, infection of bone fractures, infection of prosthetic implants (such as an artificial hip joint), infections elsewhere in the body that reach the bones via the bloodstream, a primary infection of the blood (septicaemia). Risk factors that may increase a person's susceptibility to osteomyelitis include: long term skin infections, inadequately controlled diabetes, poor blood circulation (arteriosclerosis), risk factors for poor blood circulation, which include high blood pressure, cigarette smoking, high blood cholesterol and diabetes, immune system deficiency, prosthetic joints, the use of intravenous drugs, sickle cell anaemia, and cancer. Treatment for osteomyelitis depends on the severity but may include: replacement of the infected prosthetic part, surgery to clean and flush out the infected bone (debridement), skin grafts, if necessary.

In another aspect, the solution and apparatus of the invention is particularly suitable for use in conjunction with stem cell and growth factor therapy, including the use of genetically engineered cells and engineered tissue and allografts in various treatments. Using the solution or apparatus of the invention to disinfect tissue before, during or after addition of cells or growth factors, maintains the viability of the cells and integrity of the growth factors, while killing the unwanted microbes.

In certain embodiments of the method of the invention (e.g., for treating wounds, bone, tissue, or surgical cavity), the solution is an electrolyzed saline solution, such as an electrolyzed saline solution containing hypochlorous acid as the main active agent, as described herein. In these or other embodiments, the electrolyzed solution may contain hypochlorite. The electrolyzed saline solution may have a pH of from 4 to 7, or a pH of from 4.5 to 6.5, or a pH of from 5.0 to 5.8, or about 5.4.

In one embodiment, the method comprises providing an electrolyzed saline solution and a wound dressing described herein. The wound dressing is applied to the wound site and the electrolyzed saline solution is infused. In accordance with this embodiment, the solution may be dispensed to the wound by gravity or by employing a pump. The method may further comprise sensing a condition indicating the status of the wound site, the wound dressing, or the wound exudate. For example, the condition being sensed may be pH, nitric oxide level, temperature, fluid flow rate, moisture level, fluid pressure, or electrical conductivity of the fluid. Responsive to the condition sensed, the quantity or the quality of the electrolyzed saline solution being provided to the wound dressing is adjusted. For example, the flow rate or the pH of the solution may be adjusted. A controller may be used to receive information about the condition being sensed and perform the adjustments.

In certain embodiments, the method may further comprise extracting the wound exudate from the wound site. The extraction may be performed by vacuum suctioning of the wound dressing. In certain embodiments, the method may further comprise recirculating the wound exudate to the wound site. The wound exudate may be recirculated into the electrolyzed saline solution or the wound dressing and the recirculation process may be manual or automated The wound exudate may be processed (e.g., filtered) prior to being recirculated.

### IV. Exemplary Embodiments

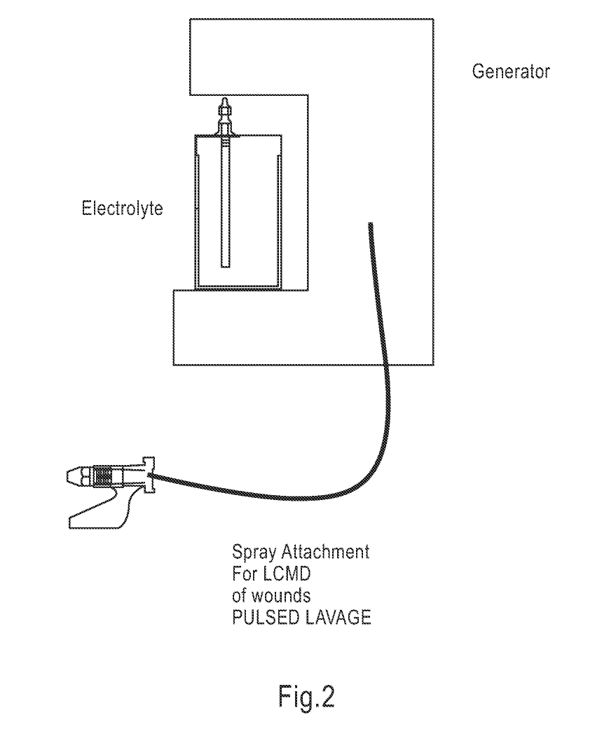

FIG. 2 shows an embodiment of the invention employing pulsed lavage to infuse a wound with an electrolyzed solution. Dry electrolyte is provided and an electrolyzed solution prepared using a self-contained generator. A spray attachment in fluid communication with the system may be used for pulsed lavage of the wound with the electrolyzed solution.

FIG. 3 shows an embodiment of the invention using a wound dressing with pulsed lavage and an electrolyzed solution. Dry electrolyte is provided and an electrolyzed solution prepared using a self-contained generator. The electrolyzed solution is supplied to the wound, and contained around the wound with an occlusive dressing that contains a mechanism for pulsed lavage. Suction may also be applied for negative pressure therapy and for removing exudate and wound waste.

FIG. 4 shows an apparatus 10 according to an embodiment of the present invention. Apparatus 10 comprises an electrochemical generator 20 having an electrolytic cell system 22. Electrolyzed solution is generated by electrolytic cell system 22 and stored in a storage container 26 until solution is demanded. The solution is pumped out of storage container 26 by a dispensing pump 28 and dispensed through a dispenser 24, which is connected to supply conduit 82 (shown in FIG. 6) of tube 80 (FIG. 7). Dispensing pump 28 is controlled by a controller 40. Controller 40 also controls the electrolytic cell system 22.

In certain embodiments, the apparatus further comprises one or more tubings between various components of the apparatus. The tubings may have one or more channels or conduits for the transportation of fluid or to contain transmission lines. In certain instances, the apparatus may comprise a plurality of tubings. In certain instances, two or more of the tubings may be combined into a single unit.

The fluid conduits can carry fluid to or from the various components of the apparatus. For example, the tubing may have a conduit for transporting the electrolyzed solution from the electrochemical generator to the wound dressing and/or wound exudate from the wound dressing to a vacuum canister. The tubings may have another conduit for transporting another biologically active substance, such as a drug or a byproduct (e.g., NaOH) of the electrochemical generator. The transmission line may connect a sensor to a controller. For example, the transmission line may connect a sensor in the wound dressing to a controller in the electrochemical generator. The transmission lines may be any communication line capable of carrying a signal, such as electrical wire or fiber optic cable.

In an embodiment as shown in FIGS. 7A, a first tubing 80 connects an electrochemical generator 20 to a vacuum canister 60 (FIG. 4) and a second tubing 90 (FIG. 7B) connects the vacuum canister 60 to a wound dressing 100 (FIGS. 4 and 6). The first tubing 80 and second tubing 90 are in communication with each other. First tube 80 and second tube 90 carry a supply conduit 82 and a communication channel 84 containing a transmission line 86. Second tube 90 additionally carries an exudate drainage conduit 92. Supply conduit 82 in first tube 80 and second tube 90 are in fluid communication. The proximal end of supply conduit 82 in first tube 80 is connected to dispensing outlet 24 of electrochemical generator 20 (see FIG. 4). The distal end of supply conduit 82 in second tube 90 is disposed in reservoir layer 112 (see FIG. 6) of wound dressing 100. The distal end of supply conduit 82 also has a one-way valve 130 to prevent backflow and several openings to allow outflow of the solution into reservoir layer 112.

The proximal end of exudate drainage conduit 92 is disposed in contact layer 116 of wound dressing 100 and the distal end is in communication with vacuum canister 60. The proximal end of exudate drainage conduit 92 also has several openings to allow the extraction of wound exudate. Transmission line 86 in tubes 80 and 90 are in communication with each other. One end of transmission line 86 in tube 80 is in communication with a controller 40 and the other end of transmission line 86 in tube 90 is in communication with a sensor 30.

In certain embodiments, the apparatus further comprises a vacuum canister which collects the wound exudate. The wound exudate may be drawn into the vacuum canister by vacuum suction created by a vacuum source, such as a motorized suction pump. The vacuum source may be combined with the vacuum canister or it may be external to the vacuum canister, such as a portable electrically-powered pump or suction supplied through a wall outlet as commonly provided in hospitals. Alternatively, the vacuum source may be combined with the electrochemical generator to form a single unit.

The vacuum canister can be in fluid communication with other components of the apparatus via one or more tubings. For example, the vacuum canister may be coupled to a tubing in communication with a wound dressing for suctioning wound exudate. The vacuum canister may operate independently or it may operate under the control of a controller. In some instances, a controller may operate both the vacuum canister and the electrochemical generator to coordinate the operation of these components.

In an embodiment, as shown in FIG. 4, a vacuum canister 60 comprises a receptacle container 62, an inlet port 64, and a suction port 68. Inlet port 64 is in fluid communication with exudate drainage conduit 92. Suction port 68 is in fluid communication with a suction pump 70 coupled to vacuum canister 60 via a suction tube 66. In operation, suction generated by suction pump 70 is transmitted through suction tube 66, through suction port 68, through inlet port 64, and through exudate drainage conduit 92, to the wound site. The suction draws the wound exudate and transports it to vacuum canister 60 via exudate drainage conduit 92 in tube 90. Wound exudate is collected in receptacle container 62 through inlet port 64. Suction pump 70 is controlled by controller 40 via control line 72. As an additional feature, controller 40 may coordinate both the suction of wound exudate and the delivery of electrolyzed saline solution produced by electrochemical generator 20 to optimize fluid flow dynamics or efficacy in promoting wound healing.

In certain embodiments, the apparatus may further comprise a wound exudate recirculation system that allows for the recirculation of the wound exudate to the wound site.

In an embodiment, as shown in FIG. 6, an exudate recirculation extension 120 is provided for wound dressing 100. Exudate recirculation extension 120 comprises an injection port 122, a filter 124, and an injection tubing 126. The distal end of injection tubing 126 is disposed in reservoir layer 112. In operation, wound exudate is drawn into a syringe and then injected through injection port 122. Debris, bacteria, and other contaminants are filtered-out by filter 124. The wound exudate then travels through injection tubing 126 and is delivered to reservoir layer 112, where it is recirculated to the wound site.

In an alternate embodiment, as shown in FIG. 8, an apparatus 300 comprises a source of electrolyzed saline solution 320, a wound dressing 330, supply conduit 310, drainage conduit 312, and vacuum canister 340. Electrolyzed saline solution is provided to wound dressing 330 via supply conduit 310. Facilitated by a suction pump 350, wound exudate is extracted from wound dressing 330 and drained into vacuum canister 340 via drainage conduit 312. A recirculation pump 360 withdraws wound exudate from vacuum canister 340 and recirculates it. As shown by the broken arrows, the wound exudate may be recirculated into the electrolyzed saline solution at various points in apparatus 300, including source 320, supply conduit 310, or wound dressing 330.

Elecrolyte may be supplied in bottles, such 3 liter bottles (FIG. 10A). The bottles may be placed in the apparatus of the invention to produce and deliver the electrolyzed solution, and provide a convenient manner for hospital reimbursement. The container may further employ a closed-loop electrolyte management system. The blow molded container shown in FIG. 10A has two chambers, one carrying electrolyte and another for storing waste from the wound and/or containing the waste product of the electrolysis process. RFID tags or bar code printing may be on each container to manage inventory and waste, and provide information to the controller. The electrolyte container may be designed so as to be easily positioned within the apparatus of the invention. For example, in one embodiment, placement of the electrolyte container in the apparatus produces an audible click, indicating that the container is properly inserted within the device (FIGS. 10B and C).

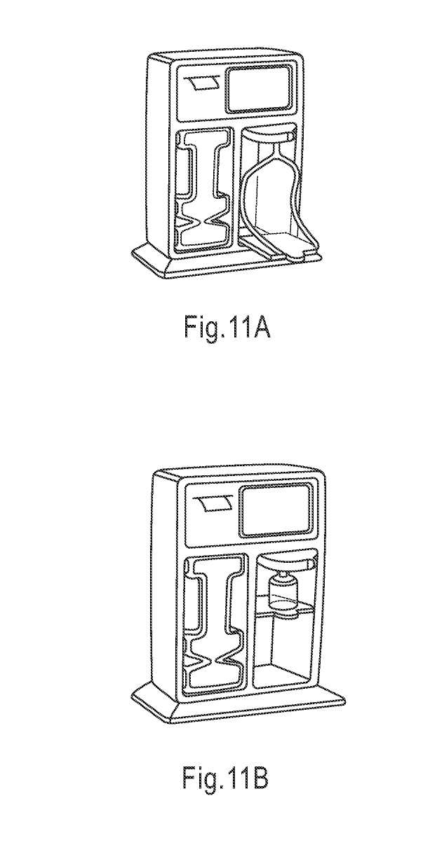

The apparatus of the invention may employ containers, for storing the electrolyzed solution, that are an ordinary part of a hospital or health care facilities' inventory, such as IV bags and 100 cc or 1000 cc bottles (FIG. 11A and b). The apparatus may employ an elevator mechanism to adapt to different sizes and shaped of containers.

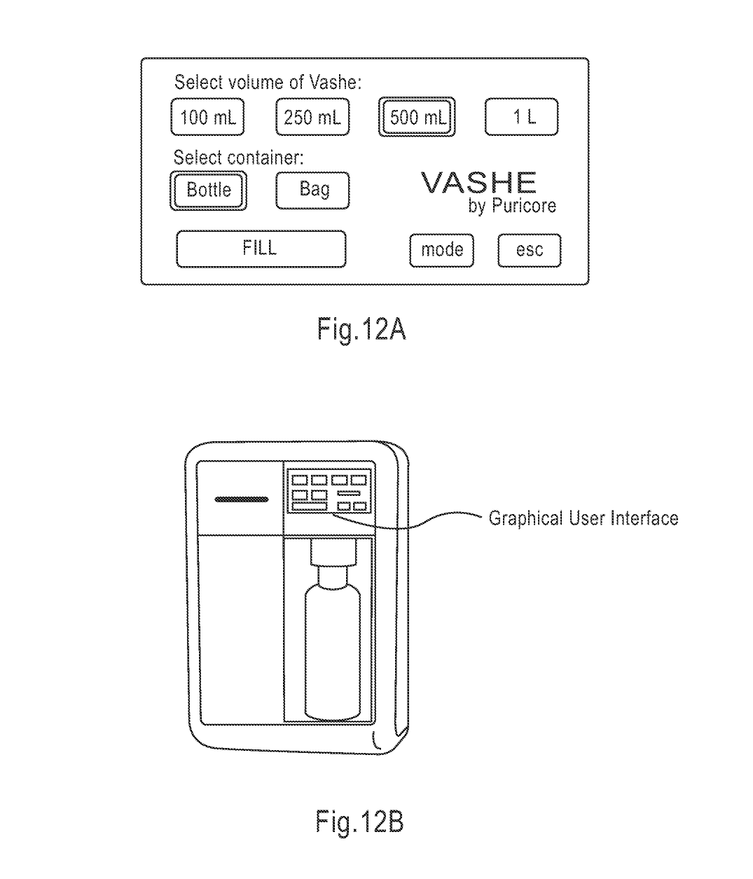

The apparatus of the invention may contain a graphical user interface to facilitate use (FIG. 12A and B). The user interface may display controls for solution volume, container type or shape, pH, AFC concentration, mode of operation or delivery (e.g. hydrosurgery, pulsed lavage, negative pressure), as well as continuous or intermittent production of electrolyzed solution.

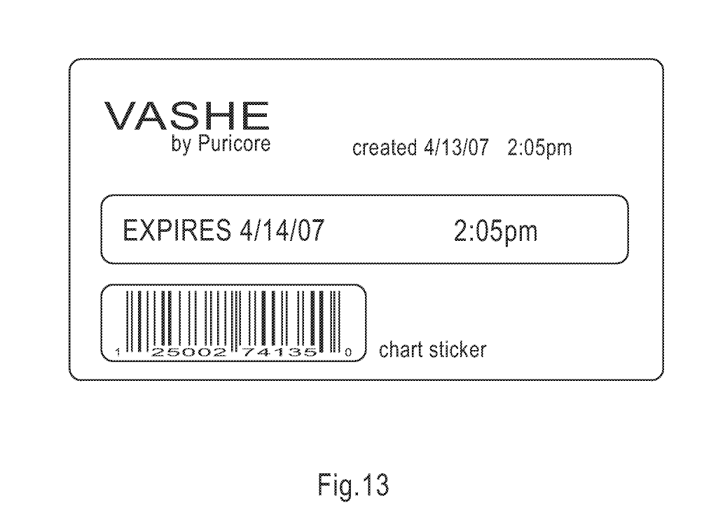

The solution prepared by the apparatus may be labeled to indicate the patient for whom the solution was prepared, and the estimated expiration date. Adhesive labels may be prepared and dispensed by the apparatus, or the apparatus may print directly on the container. (FIG. 13).

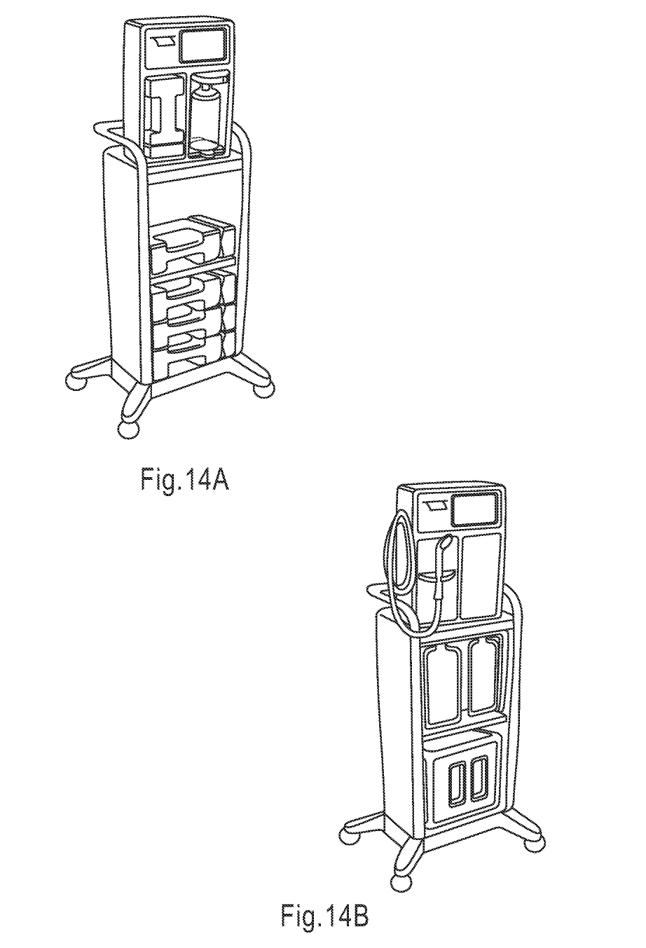

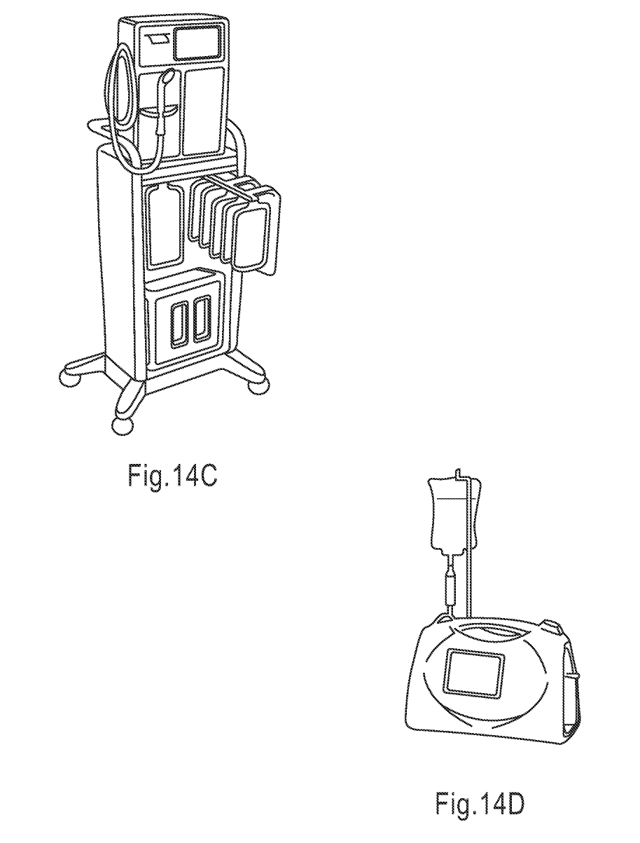

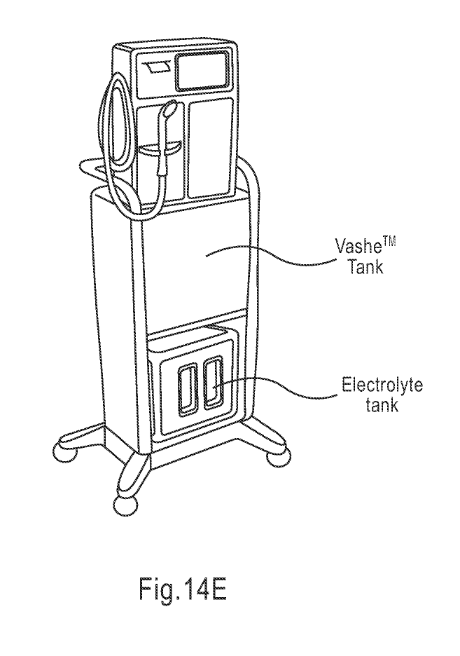

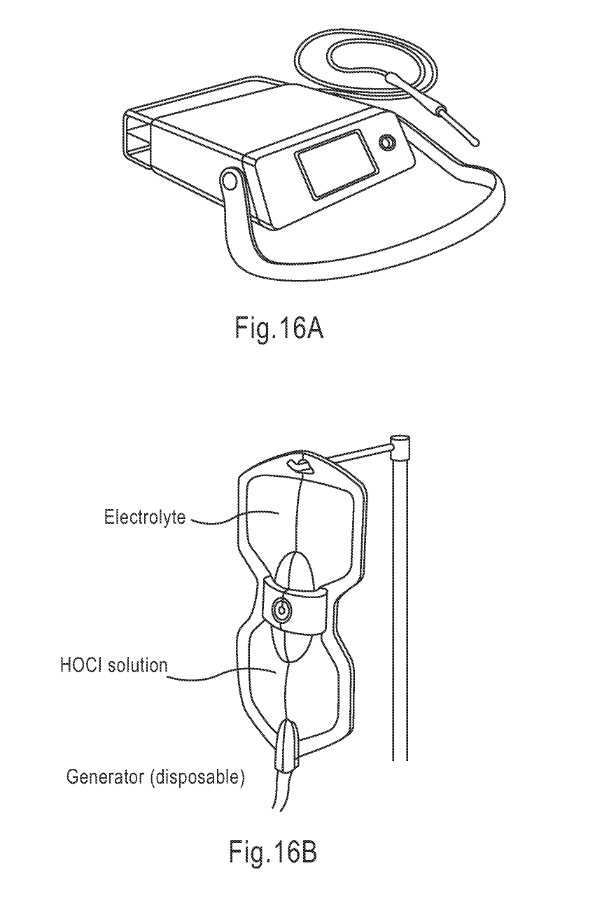

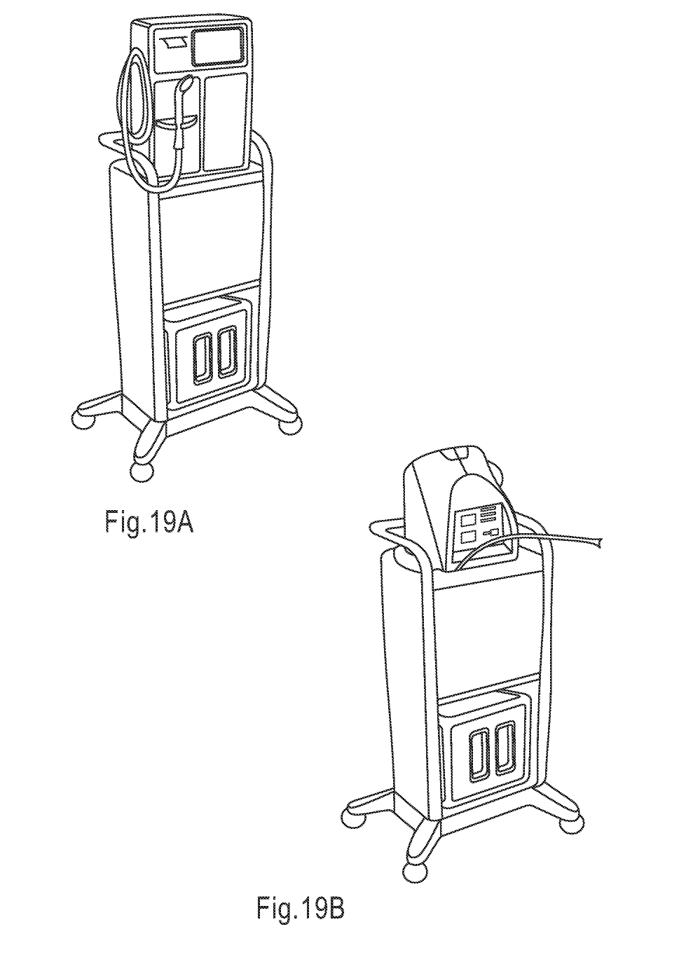

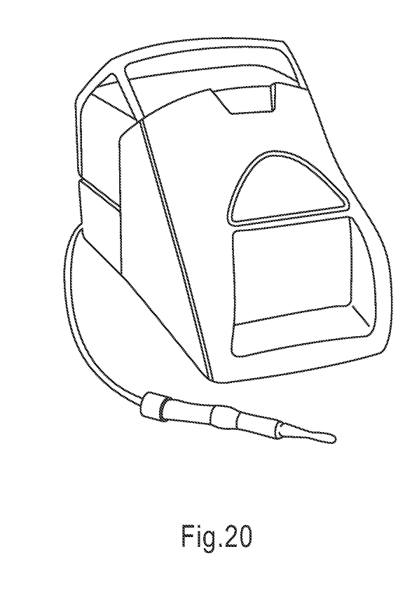

Table top or cart top units may be utilized which further provide for convenient storage of solution, electrolyte bottles, and wound dressings as described herein (FIG. 14A-E, FIGS. 19A and B, and FIG. 20). Further, as shown in FIGS. 16A and B, miniaturized systems of the invention may be used to further increase the portability of the wound care system. Such embodiments include custom IV bags containing two chambers separated by an electrochemical generator. The first chamber containing an electrolyte solution, and the second chamber containing a reservoir for the solution once electrolyzed. In these embodiments, the system may be connected to a wound dressing as disclosed herein, and may be disposable.

The foregoing description and examples have been set forth merely to illustrate the invention and are not intended as being limiting. Each reference, patent or patent application referred to herein is incorporated by reference in its entirety for all purposes. Each of the disclosed aspects and embodiments of the present invention may be considered individually or in combination with other aspects, embodiments, and variations of the invention. Further, while certain features of embodiments of the present invention may be shown in only certain figures, such features can be incorporated into other embodiments shown in other figures while remaining within the scope of the present invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention.

### EXAMPLES

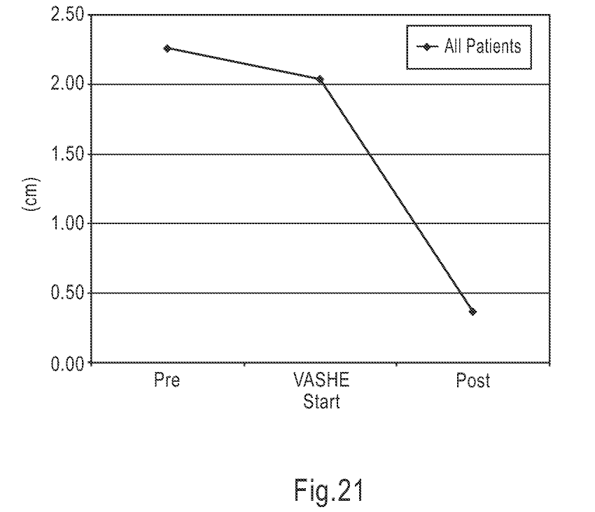

142 patients were treated with HOCI solution at the Centers for Comprehensive Wound Care and Hyperbaric Oxygen Therapy (Aurora Health Care, St. Luke's Medical Center, Milwaukee, Wis.). The regimen involved an HOCI soak followed by a vigorous HOCl solution scrub. Wound healing trajectories were analyzed in these patients prior to the initiation of HOCl treatment, and then recalculated 6 weeks afterward. All patients healed or showed significant improvement after treatment using the above regimen. No adverse effects were seen. See FIG. 21.

Thus, debridement and disinfection with HOCl solution offers benefits for improved wound care.

### EXAMPLE 1

### Treatment of Chronic Venous Ulcers

Patients who failed to heal with prior conventional therapy including topical anti-microbial agents and compression therapy were treated with HOCl solution. The treatment regimen involved an HOCl solution soak followed by a vigorous scrub with the solution. All patients healed or showed significant improvement after treatment using the above regimen. No adverse effects were seen.

One case involved a 44 year old male with a history of venous insufficiency wounds to bilateral medial ankles, which was re-occurring despite aggressive compression, topical antibiotics and local wound care. Wounds had been present for 7-8 months prior to treatment with the HOCl solution.

A marked increase in the slope of the curve (decreased wound volume) was found with the use of HOCl wound cleanser.

### EXAMPLE 2

### Treatment of Necrotizing Fasciitis

Necrotizing Fasciitis (NF) is a rare, life-threatening bacterial infection of the skin, subcutaneous tissue, and superficial fascia, associated with a mortality rate of 20-60%. During the past three decades, the incidence of NF has increased and now is estimated at 0.4 cases per 100,000. Pathergy can be due to a single species of bacteria or a polymicrobial process. Historically the treatment of NF included aggressive surgical debridement, systemic antibiotics and adjunctive care including hyperbaric oxygen therapy (HBOT) and local wound care.

A series of patients with significant wounds following surgical debridement of necrotizing soft tissue infections were managed with HOCI infusion and NPWT. All wounds showed clearance of infective organisms as evidenced by no further progression of tissue ischemia and necrosis, reversal of tissue cellulitis, and rapid formation of granulation tissue. Patients were surgically closed sooner after initiation of HOCI infusion therapy when compared to historical controls.

In one case, a 62 year old female with a past medical history of diabetes and obesity developed necrotizing fasciitis of her groin. The patient was treated with aggressive surgical debridement, systemic antibiotics, and hyperbaric oxygen therapy. Her post operative wound management included NPWT with HOCI infusion. The wound showed excellent granulation at 2 weeks and was surgically closed shortly thereafter.

In a second case, a 24 year old female developed necrotizing fasciitis of her abdominal wall post laparoscopic salpingectomy. The patient was treated with aggressive surgical debridement, systemic antibiotics, and hyperbaric oxygen therapy. Primary wound management included NPWT with HOCl infusion. Early granulation was noted and complete closure was achieved with delayed primary closure 16 days after initial debridement.

In conclusion, HOCl infusion is effective for post-surgical management of patients with necrotizing soft tissue infections and highly contaminated wounds, and in combination with NPWT. HOCl infusion with NPWT achieves excellent wound granulation, allowing for earlier surgical closure and hospital discharge.

### EXAMPLE 3

### Treatment of Surgical Wounds

Wound closure is optimally achieved when an orderly transition from inflammation through proliferation and remodeling is realized. Negative Pressure Wound Therapy (NPWT) is effective in maximizing the formation of granulation tissue. The wound specialist is then challenged with the decision of what product to transition to once the end points of NPWT have been attained, complete wound base granulation with minimal depth and undermining. The ideal therapy after NPWT would maintain the presence of the fibroblast, limit bioburden and provide proper wound moisture to promote neoepithelialization.

A series of patients with non-healing surgical wounds were treated with NPWT until the wound bases were well granulated with superficial depth, without undermining or tunneling. These patients were then transitioned to topical therapy with hypochlorous acid. Healing rates were compared to standard moist wound healing regimens. The following cases are presented to illustrate the wound healing trajectories that can be attained with the use of HOCl as the primary topical agent after wounds have reached an endpoint for management with NPWT.

In one case, a female (age 53) with a past medical history of diabetes, diabetic neuropathy, and hypertension developed a left diabetic foot ulcer with underlying osteomyelitis. The patient (Wagner's III DFU) was managed with debridement (partial foot amputation), intravenous antibiotics and then treated postoperatively with hyperbaric oxygen therapy and NPWT. The patient was transitioned to HOCl therapy after reaching the end point of NPWT. Wound closure was obtained in six weeks with HOCl treatment.

In a second case, a 42 year old male underwent excision of an ectopic bone of his right hip. After a several month failure to heal the patient was referred to the wound care center. The patient was treated with NPWT for 3 weeks and then transitioned to HOCI daily dressing changes. Wound closure was again obtained in six weeks with HOCl treatment.

1. A treatment apparatus comprising:
a reservoir holding a treatment solution and/or a generator producing a treatment solution, wherein the treatment solution comprises hypohalous acid as an active agent;
a mechanism for delivering, and/or controlling the delivery of, the treatment solution from the reservoir or generator to a wound site or tissue; and
a device for infusing the wound or tissue with the solution.

2. The apparatus of claim 1, further comprising a device for applying and/or controlling the application of negative pressure to the wound or tissue, wherein the device for applying and/or controlling negative pressure is physically and functionally combined with the device for infusing the wound or tissue with the solution.

3. The apparatus of claim 1, wherein the solution is an electrolyzed saline solution.

4. The apparatus of claim 3, wherein the electrolyzed saline solution contains essentially hypochlorous acid as an active agent for wound therapy.

5. The apparatus of claim 3, wherein the electrolyzed saline solution has a pH of from 4 to 7.

6. The apparatus of claim 3, wherein the electrolyzed saline solution has a pH of from 4.5 to 6.5.

7. The apparatus of claim 3, wherein the electrolyzed saline solution has a pH of from 5.0 to 5.8.

8. The apparatus of claim 3, wherein the apparatus has a self-contained generator for producing the electrolyzed saline solution.

9. The apparatus of claim 1, wherein the apparatus is portable.

10. The apparatus of claim 1, wherein the solution is dispensed in the form of a liquid, cream, gel, or foam.

11. The apparatus of claim 1, wherein the device is an occlusive wound dressing that delivers the solution by pulsatile lavage or with ultrasound.

12. The apparatus of claim 1, further comprising a mechanism that heats and maintains the temperature of the solution at about body temperature.

13. The apparatus of claim 1, wherein the device for delivering the solution to the wound or tissue is a device for hydrosurgery, pulsatile lavage, and/or ultrasound.

14. A method for treating a wound comprising infusing a wound or tissue with a hypohalous acid solution before, during, or after negative pressure wound therapy.

15. A method for reducing wound bioburden comprising infusing a wound or tissue with a hypohalous acid solution before, during, or after negative pressure wound therapy.

16. The method of claim 14, wherein the wound is colonized or infected with a drug resistant microorganism.

17. The method of claim 16, wherein the hypohalous acid solution is applied so as to reduce the number of said drug resistant microorganisms by about 6 logs.

18. The method of claim 14, wherein the hypochlorous acid solution is at body temperature.

19. The method of claim 14, wherein the infusion of the wound or tissue with hypohalous acid is coordinated with the application of negative pressure.

20. The method of claim 14, wherein negative pressure is applied before infusion of the wound with the hypohalous acid solution.

21. The method of claim 14, wherein the hypochlorous acid solution is an electrolyzed saline solution.

22. The method of claim 21, wherein the electrolyzed saline solution contains from about 0.2 to about 2% w/v NaCl.

23. The method of claim 21, wherein the electrolyzed saline solution contains essentially hypochlorous acid as an active agent for wound or tissue therapy.

24. The method of claim 21, wherein the electrolyzed saline solution has a pH of from 4 to 7.

25. The method of claim 21, wherein the electrolyzed saline solution has a pH of from 4.5 to 6.5.

26. The method of claim 21, wherein the electrolyzed saline solution has a pH of from 5.0 to 5.8.

27. The method of claim 21, wherein the electrolyzed saline solution is generated and supplied to the wound using an apparatus comprising:
a generator producing the electrolyzed saline solution;
a pump delivering the solution to a wound site; and
a device for delivering the solution to the wound by pulsatile lavage, hydrosurgery, and/or ultrasound.

28. The method of claim 27, wherein the apparatus further comprises a means for applying and controlling negative pressure at the site of a wound or tissue.

29. The method of claim 14, wherein the wound is a chronic venous ulcer, necrotizing fasciitis, partial thickness bum, full thickness bum, or a post-surgical wound.

30-33. (canceled)

34. A method for the treatment of a chronic wound using a wound treatment regimen, the regimen comprising:
a) applying reduced pressure to the wound; and
b) treating the wound with a substantially non-toxic, non-irritating hypochlorous acid composition while applying reduced pressure to the wound.

35. The method of claim 34, wherein the treatment of the hypochlorous acid composition to the wound is performed before application of reduced pressure to the wound.

36. The method of claim 34, wherein the hypochlorous acid composition treatment of the wound is done after application of reduced pressure to the wound.

37. The method of claim 34, wherein the hypochlorous acid composition treatment and application of reduced pressure to the wound is done intermittently.

38. The method of claim 34, wherein the treatment with the hypohalous acid composition is performed by an automated delivery system.

39. The method of claim 34, wherein the hypohalous acid composition has a pH of from about 4 to 7.

40. The method of claim 34, further comprising, treating the wound with saline.
